(19) United States
(12) Patent Application Publication
(10) Pub. No.: US 2025/0339105 A1
(43) Pub. Date: Nov. 6, 2025

(54) SYSTEM AND METHOD FOR DIAGNOSIS AND NOTIFICATION REGARDING THE ONSET OF A STROKE

(71) Applicant: YALE UNIVERSITY, New Haven, CT (US)

(72) Inventors: Hitten P. Zaveri, New Haven, CT (US); Kevin N. Sheth, Madison, CT (US); Ronald R. Coifman, North Haven, CT (US); Hyeon Ki Jeong, Duluth, GA (US); Denis Filatov, Madrid (RS)

(73) Assignee: ALVA HEALTH, INC., Dallas, TX (US)

(21) Appl. No.: 19/266,917

(22) Filed: Jul. 11, 2025

Related U.S. Application Data
(63) Continuation-in-part of application No. 17/308,447, filed on May 5, 2021, now Pat. No. 12,357,244.
Application No. 17/308,447 is a continuation of application No. 16/069,548, filed on Jul. 12, 2018, now Pat. No. 11,020,059.
(60) Provisional application No. 62/277,645, filed on Jan. 12, 2016.

(51) Int. Cl. A61B 5/00; A61B 5/0205; A61B 5/024; A61B 5/0533; A61B 5/11; G16H 10/60

(52) U.S. Cl. CPC A61B 5/7282; A61B 5/0022; A61B 5/1114; A61B 5/1118; A61B 5/6824; A61B 5/6828; A61B 5/6829; A61B 5/7275; A61B 5/746; G16H 10/60; A61B 5/02055; A61B 5/02438; A61B 5/0533; A61B 5/1122; A61B 5/4064; A61B 2505/01; A61B 2505/09; A61B 2562/0219

(57) ABSTRACT

A real-time automated method to diagnose and/or detect stroke and engage the patient, care-takers, emergency medical system and stroke neurologists in the management of this condition includes the steps of continuously measuring natural limb activity, conveying the measurements to a cloud based real-time data processing system, identifying patient specific alert conditions, and determining solutions for acting upon needs of the patient. The system by which the method is implemented includes at least one body worn sensor continuously measuring natural limb activity and a patient worn data transmission device conveying the measurements to a cloud based real-time data processing system that identifies patient specific alert conditions and determines solutions for acting upon needs of the patient. In an example solution, motion data that reflects upper limb movements of a user is received from one or more sensors, specific changes in user movement are determined by estimating several quantitative signal features, and the results are input into a machine learning model to detect if the user's movements reflect a change due to the occurrence of a stroke. The quantitative features and the machine learning model determine the degree of motor deficit induced by a stroke as reflected by changes in time-series measures of signal magnitude, variability, complexity, and interrelation. The solution operates in two distinct modes, one by continuously monitoring subject activity and the second by evaluating short duration data segments when the subject is performing prescribed movement tasks. In both modes the solution detects if the user has suffered a stroke and estimates a motor deficit score to determine the severity of the stroke.

### CROSS REFERENCE TO RELATED APPLICATION

This application is a continuation-in-part of U.S. patent application Ser. No. 17/308,447, entitled “SYSTEM AND METHOD FOR DIAGNOSING AND NOTIFICATION REGARDING THE ONSET OF A STROKE,” filed May 5, 2021, which is a continuation of U.S. patent application Ser. No. 16/069,548, entitled “SYSTEM AND METHOD FOR DIAGNOSING AND NOTIFICATION REGARDING THE ONSET OF A STROKE,” filed Jul. 12, 2018, which is a 371 of PCT Application No. PCT/US17/13149, entitled “SYSTEM AND METHOD FOR DIAGNOSING AND NOTIFICATION REGARDING THE ONSET OF A STROKE,” filed Jan. 12, 2017, which claims the benefit of U.S. Provisional Patent Application Ser. No. 62/277,645, entitled “SYSTEM AND METHOD FOR DIAGNOSING AND NOTIFICATION REGARDING THE ONSET OF A STROKE,” filed Jan. 12, 2016, all of which are incorporated herein by reference.

### BACKGROUND OF THE INVENTION

### 1. Field of the Invention

The invention relates to a system and method for diagnosis, detection, and notification regarding the onset of a stroke.

### 2. Description of the Related Art

Ischemic stroke is the fifth leading cause of death, and a leading cause of disability in the United States, affecting 700,000 Americans every year. Even though intravenous (IV) tissue plasminogen activator (tPA) has been an FDA approved therapy since 1995, thirty years later, less than 10% of eligible patients receive this therapy.

IV tPA is a treatment for acute ischemic stroke with proven benefit. tPA is a protein involved in the breakdown of blood clots. It is a serine protease found on endothelial cells, the cells that line the blood vessels. As an enzyme, it catalyzes the conversion of plasminogen to plasmin, the major enzyme responsible for clot breakdown. While IV tPA can be effectively used to treat strokes and there is robust population level data to show that every minute of delay results in a worse outcome, the rate of timely tPA administration to ischemic stroke patients is exceedingly low.

Treatment with IV tPA in a 3-4.5 hour time-window after the onset of a stroke can increase favorable outcome. Rapid evaluation with computed tomography (CT) or MR imaging in the emergency department (ED) is required to rule out hemorrhagic stroke before giving tPA. Overall, only 22% of all stroke patients in the U.S. arrive in the ED within the first several hours of stroke onset, and recent epidemiological studies indicate that approximately 7% are treated with IV tPA. Mechanical thrombectomy may be effective in a subset of these early presenting patients who have a large vessel occlusion identified on angiography. Thrombectomy also has a time-treatment interaction with earlier treatment resulting in improved outcomes. Subsequent management is largely supportive, with a focus on identifying the etiology to prevent stroke recurrence. Because pain is usually absent from symptoms of stroke and because patients are neurologically disabled, patients routinely face a delay in timely diagnosis. Which in turn affects both their potential eligibility for these acute therapies and when received, the potential efficacy they may derive from its administration.

Two strategies have been employed to date in order to reach patients faster. First is the “drip and ship” strategy where patients arrive at community hospitals, are connected to stroke neurologists through telemedicine and then transferred while tPA is being administered. The second, more recent approach is to place a CT scanner in an ambulance to bring the emergency room to the patient. Both of these approaches are inefficient, expensive, and have not made a significant impact in tPA administration rates.

Despite massive public health campaigns, identifying symptoms of stroke and activating emergency response systems within the 4.5 hour time window for effective IV tPA treatment continues to remain a major challenge. While the FDA recently approved tenecteplase (TNKase), which is a newer version of thrombolysis that is starting to replace tPA and is a little better, earlier this year, the existing challenges still remain. The solution, real time detection of stroke, remains a major public health goal with financial consequences of billions of dollars and a reduction in disability or death for tens of thousands of patients.

It is also appreciated that the human brain has distinct cortical and sub-cortical areas in each hemisphere which govern motor activity. The main cortical areas involved in motor activity are the primary motor cortex, pre-motor cortex, and the primary somatosensory cortex which are all located on the lateral convexity of each hemisphere. Other areas involved in voluntary movements include the basal ganglia, cerebellum, pedunculopontine nucleus, and subcortical motor nuclei and the parts of the nervous system which relay signals to muscles and to sensory cortex. The motor cortex on the left hemisphere controls activity on the right side of the body and the motor cortex on the right hemisphere controls motor activity on the left side of the body. These different cortical and subcortical areas of the brain work in conjunction with each other and with other parts of the brain, the peripheral nervous system, sensory system, balance and coordination centers, and muscles to produce the complex movements which are articulated by human beings. The mechanism by which this control, communication, and coordination is achieved is not fully known. It is, however, understood that these parts of the brain and body communicate and act together as a motor control system to create the full range of human movement which is expressed.

Each individual executes movement in a manner which is governed by that individual's motor control system. That is, the movement is the result of the above-described areas within the brain, the interaction of those areas with the peripheral nervous system and the muscles which are activated. The movement expressed encompasses these control and actuator aspects as well as the feedback the brain receives from the motor and sensory systems and balance and coordination centers. The movement performed by an individual is specific to that individual, and reflects key aspects of the individual, for example, age, handedness, sex, and individual strengths and weaknesses, including prior training if any. A stroke or an injury to the brain, be it in the motor areas, or in areas which are outside the motor areas, for example the visual areas, will affect the motor control system and the movement expressed. This influence will be direct in those instances where the motor areas are directly affected by the stroke, and indirect in cases where other brain areas (e.g., visual areas) are impacted. Whether the stroke directly or indirectly affects the motor areas of the brain, it will affect the movement signatures of the individual. When a stroke affects primary motor areas, it will impact the movement articulated by the limbs on the contralateral side of the body. The result of a stroke will be a deficit in the richness of the movement which can be expected to be proportional to the severity of the stroke and how directly or indirectly it affects the motor area of the brain. If, for example, a stroke occurs outside the primary motor areas, there will still be a change in the individual's movements because the motor control system draws upon a large extent of the brain as described above and the multiple different functions of the brain impact the movement of the individual. That is, we can expect if a stroke is minor or if it impacts areas outside those areas responsible for movement the resulting deficit in the richness of the movements articulated will be small, while if the stroke is large or directly affects brain areas responsible for movement the resulting deficit in the richness of the movements articulated will be considerable.

Currently, most patients are delayed in their presentation to the hospital because of lack of awareness of having suffered a stroke or failure in activating emergency systems. Early detection is the most significant problem in acute stroke care. A further contributor to the challenge is incorrect symptom recognition with most stroke emergencies being misidentified by emergency medical technicians. Bridging this gap with a fast, easy, and objective solution to identify stroke would have a significant impact, because many more patients could access therapies in a window that is known to improve outcomes.

The present invention seeks to solve this major gap in clinical practice, which is real-time diagnosis and/or detection of ischemic and hemorrhagic stroke and acute central nervous system injury. The development of a real-time automated system and method to diagnose and detect stroke and to engage the emergency medical system and stroke neurologists has the potential to dramatically shorten the time to definitive therapy for patients with stroke.

### SUMMARY OF THE INVENTION

It is, therefore, an object of the present invention to provide a real-time automated method to diagnose and/or detect stroke and engage the patient, care-takers, emergency medical system and stroke neurologists in the management of this condition. The method includes the steps of continuously measuring natural limb activity, conveying the measurements to a cloud based real-time data processing system, identifying patient specific alert conditions, and determining solutions for acting upon needs of the patient.

It is also an object of the present invention to provide a method wherein the step of determining solutions includes providing for notification of potential stroke syndromes in real-time and activating acute stroke protocols.

It is another object of the present invention to provide a method wherein the step of identifying includes establishing patient specific limb activity signature through the aggregation of continuously sampled data acquired over minutes, hours, days, weeks, and months.

It is another object of the present invention to provide a method that analyzes task-based data.

It is a further object of the present invention to provide a method wherein the step of continuously measuring natural limb activity includes positioning two or four body worn sensors on the limbs of the patient.

It is also an object of the present invention to provide a method wherein the step of conveying the measurements includes adding a time-stamp to a data stream generated by continuously monitoring limb activity.

It is another object of the present invention to provide a method wherein the step of determining solutions includes identifying treatment protocols.

It is a further object of the present invention to provide a method wherein the treatment protocols include activation of the emergency medical response system, transport of the patient to the nearest Neurocritical Care Unit or emergency room for rapid evaluation and treatment.

It is also an object of the present invention to provide a method wherein the step of identifying patient specific alert conditions includes creating diffusion maps representative of the patient's limb movement.

It is also an object of the present invention to provide a method wherein the step of identifying patient specific alert conditions includes considering measures of signal magnitude, variability, complexity, and interrelation.

It is another object of the present invention to provide a method for determining whether the current limb movements of the patient are within expected parameters, in comparison to a previously determined patient specific limb activity signature, wherein the step of identifying patient specific alert conditions includes continuously processing limb activity and sensor data.

It is a further object of the present invention to provide a method including the step of quantifying the resulting magnitude of the change due to stroke.

It is also an object of the present invention to provide a method including the step of quantifying the degree of success in restoring function during rehabilitation.

It is a further object of the present invention to provide a real-time automated system to diagnose and/or detect stroke and engage the patient, care-takers, emergency medical system and stroke neurologists in the management of this condition. The system includes at least one body worn sensor continuously measuring natural limb activity and a patient worn data transmission device conveying the measurements to a cloud based real-time data processing system that identifies patient specific alert conditions and determines solutions for acting upon needs of the patient.

It is also an object of the present invention to provide a system wherein the data processing system establishes a patient specific limb activity signature through the aggregation of continuously sampled data acquired over minutes, hours, days, weeks, and months.

It is a further object of the present invention to provide a system including a plurality of body worn sensors shaped and dimensioned to be worn on limbs of the patient.

It is another object of the present invention to provide a system including two or four body worn sensors shaped and dimensioned to be worn on limbs of the patient.

It is a further object of the present invention to provide a system wherein the patient worn data transmission device adds a time-stamp to a data stream generated by at least one body worn sensor.

It is also an object of the present invention to provide a system wherein the data processing system adds a time-stamp to data conveyed by the patient worn data transmission device.

It is another object of the present invention to provide a system wherein at least one body worn sensor includes a motion tracking device.

It is a further object of the present invention to provide a system wherein the data processing system identifies treatment protocols.

It is also an object of the present invention to provide a system wherein the treatment protocols include activation of the emergency medical response system, transport of the patient to the nearest Neurocritical Care Unit or emergency room for rapid evaluation and treatment.

It is another object of the present invention to provide a system method wherein the data processing system includes an acquisition system, an analysis system, and a patient management system.

It is a further object of the present invention to provide a system wherein the analysis system creates diffusion maps representative of the patient's limb movement.

It is also an object of the present invention to provide a system wherein the analysis system continuously processes limb activity and sensor data, and determines, in comparison to a previously determined patient specific limb activity signature if the current limb movements of the patient are within expected parameters.

It is another object of the present invention to provide a system wherein the data processing system operates with an understanding that greater severity of stroke is associated with greater resultant loss in richness of movement signals.

It is a further object of the present invention to provide a system wherein the data processing system determines a degree of deficit resulting from a stroke.

It is also an object of the present invention to provide a system including a machine learning model determining a degree of motor deficit induced by a stroke as reflected by changes in time-series measures of signal magnitude, variability, complexity, and interrelation.

It is another object of the present invention to provide a system wherein the system operates in two distinct modes, a first mode continuously monitoring subject activity and a second mode evaluating short duration data segments when a subject is performing prescribed movement tasks.

It is a further object of the present invention to provide a system including a computational method and machine learning models.

It is also an object of the present invention to provide a system wherein the computational method and the machine learning models operate in two different modes, analyzing continuous, long-term monitoring sensor data or analyzing finite, task-specific sensor data.

It is another object of the present invention to provide a system wherein the computational method estimates features by processing data, and raw data and the features are input into the machine learning models for training to produce optimized machine learning models and into the optimized machine learning models to classify a subject state.

It is a further object of the present invention to provide a system wherein the machine leaning models identify activities of daily living, distinguish between normal and stroke states, and determine severity of a stroke.

It is also an object of the present invention to provide a system wherein the system tracks an ensemble of diffusion geometry measurements and other time-series measures of signal magnitude, variability, complexity, and interrelation determined from measurements of the body-worn sensor.

It is another object of the present invention to provide a system wherein the data are analyzed using diffusion maps, a manifold-learning machine learning (ML) method, and time-series analysis measures.

It is a further object of the present invention to provide a system wherein the time-series analysis measures comprise non-linear energy (NLE) as a measure of signal magnitude, approximate entropy (ApEn), standard deviation, detrended fluctuation analysis (DFA) and spectral entropy as measures of signal variability and complexity, and coherence and cross-ApEn as measures of interrelationship.

It is also an object of the present invention to provide a system wherein multiple machine learning models are created for detecting activities of daily living, detecting stroke, determining severity of stroke from continuous data, and determining severity of stroke from task-based data.

It is another object of the present invention to provide a system wherein motor deficit assessment is achieved using a deep neural networks to process sequential data to detect motor asymmetry or motor deficit difference between affected and unaffected arms.

It is a further object of the present invention to provide a system wherein the system detects stroke and activities of daily living, and quantifies severity of motor deficit from tasks.

It is also an object of the present invention to provide a system wherein synchronization is achieved by considering a smartphone clock to be a master clock and constantly measure and correct wearable device clock drift with respect to the smartphone clock.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1 and 2 are schematics of the present system and method for detection and notification regarding the onset of a stroke.

FIG. 3 is a schematic showing the housing and sensor electronics of a body worn sensor in accordance with the present invention.

FIGS. 4A and 4B are schematics of first and second embodiments of the body worn sensor.

FIG. 5 is a schematic of a patient worn data transmission device in accordance with the present invention.

FIG. 6 is a schematic of a cloud based real-time data processing system in accordance with the present invention.

FIG. 7 shows diffusion maps of a pair of trial datasets for different runners.

FIG. 8 shows diffusion maps depicting two trajectories produced by the same runner.

FIG. 9 is a chart comparing various trials from runners.

FIG. 10 is a graph showing the average absolute acceleration measured from the left and right arm or hand of stroke patients (n=5; shown with a star symbol) and normal subjects (n=6; shown as a circle) measured while the individuals performed the following simple task: “cross your arms over each other five times”. The acceleration values measured from each arm or hand were averaged over the X, Y and Z axis. The results for the right arm or hand are plotted as the ordinate (Y axis), and the results for the left arm or hand are plotted as the abscissa (X axis). A linear classifier boundary is shown as a dashed line. A perfect classification results from the classification boundary. That is, there is complete separation between stroke patients and normal subjects.

FIG. 11 shows exemplary diffusion maps calculated from acceleration values measured from two normal subjects (a,b), and two stroke patients (c,d) while the individuals were performing the following task: “cycle your fists in front of you”. The diffusion maps are shown separately for each arm or hand. Note, the diffusion maps for the normal subjects capture the circular trajectory of the motion of each arm or hand. For the patients however, the diffusion maps indicate the absence of circular motion for the left arm or hand (the side of the weakness), and a distorted trajectory for the right arm or hand. The arm or hand dynamics for the normal subjects and the stroke patients are very different and this difference is clearly captured by the diffusion maps.

FIG. 12 shows a stroke monitoring and detection system. As an example, two body worn devices are shown. Each device captures limb movement continuously producing time series representative of the user's limb movements. Also, shown are computational methods and machine learning models. The models are either to be trained and validated to produce an optimal model from the outputs of the sensors and computational methods, or they are used for real-time classification of the user's activities and detection of stroke.

FIG. 13 shows the calculation of signal features which characterize the user's limb activity.

FIG. 14 shows the determination of optimal combinations of the previously calculated signal features for accurate quantification of the user's limb motion.

FIG. 15 shows the fine-tuning and optimizing of the machine learning models once the data have been collected, preprocessed, and quantified.

FIG. 16 shows example placement of wearable devices on a human body and illustrates examples of activity.

FIG. 17 shows a wearable device and the orientation of three spatial axes, X (anterior-posterior, AP), Y (medio-lateral, ML), and Z (vertical, V) of the sensor coordinate frame.

FIG. 18 shows an example of continuously recorded acceleration data of a normal subject's upper limb movements during activities of daily living. Here, both limbs are free of motor deficit.

FIG. 19 shows an example of continuously recorded acceleration data of a stroke subject's upper limb movements during activities of daily living. Here, one of the limbs has a motor deficit.

FIG. 20 displays the continuous evaluation of a signal complexity measure, approximate entropy, over eight days (Thu-Thu) in an example normal subject. The data is plotted continuously, each subplot corresponds to a day, with the hour of the day shown on the X-axis. The Y axis represents the dimensionless complexity measure as a ratio with respect to the other limb. The dashed trace is the left limb and the solid trace is the right limb. The dashed trace (left limb) shows Left/(Left+Right), while the solid trace (right limb) shows Right/(Left+Right). Note the similarity of the signal complexity of the two limbs over multiple days.

FIG. 21 displays the continuous evaluation of signal complexity using approximate entropy over four days in an example stroke subject for the affected (dashed, left) and unaffected arm (solid, right). As in FIG. 20, the values displayed are ratios, Left/(Left+Right) and Right/(Left+Right). A clear and steady difference is noted between the two traces over the study period. These results demonstrate that the difference in signal complexity between affected and unaffected limbs in a stroke subject can be striking and persistent through wake and sleep.

FIG. 22 displays an evaluation of signal complexity using approximate entropy in healthy control (normal, N) subjects and subjects with stroke severity (motor component) ranging from NIH Stroke Scale (NIHSS)=1 to NIHSS=4 to illustrate the ability to capture the deficit introduced by a stroke. The analysis was performed for data acquired in the continuous mode. The results obtained in stroke subjects were compared to results from normal volunteers. For normal subjects (“N”) the complexity of the signals from the left arm was compared to that of the right arm as the ratio Left/Right. For stroke subjects (NIHSS=1 to NIHSS=4) the complexity of the affected limb was compared to the unaffected limb as the ratio Affected/Unaffected. In this bar plot, a value of 0.5 would indicate perfect symmetry between the two limbs. It is noted that the loss of signal complexity is increasingly more profound with increasing severity of stroke. The most profound loss of signal complexity is observed in individuals with a NIHSS of 4.

FIG. 23 illustrates the use of cross-limb coherence for multinomial classification of subjects with different levels of severity of stroke. The analysis was performed for data acquired in the continuous mode. It can be observed that the more severe the motor deficit, the more likely the cross-limb coherence has values close to 0. For example, for mild deficit the likelihood of near-zero coherence is 0.23 vs. for severe deficit it is 0.32.

FIG. 24 illustrates the use of an algorithm based on standard deviation for multinomial classification of subjects with different levels of severity of stroke. The analysis was performed for data acquired in the continuous mode. When the motor deficit increases from mild to severe, the standard deviation on the affected side of the body is more likely to have low values (with probability 0.40, 0.60, 0.75, and 0.85), while on the healthy side the estimates of standard deviation remain close to the level of mild deficit. Here, the probability of low values is almost always 0.40, rarely exceeding 0.50 even in the case of severe deficit.

FIG. 25 illustrates the use of within-limb coherence for multinomial classification of subjects with different levels of severity of stroke for the healthy side of the body. The analysis was performed for data acquired in the continuous mode. Because this side is unaffected, the distribution of coherence does not vary across the different motor deficit levels.

FIG. 26 illustrates the use of within-limb coherence for multinomial classification of subjects with different levels of severity of stroke for the affected side of the body. The analysis was performed for data acquired in the continuous mode. Because this side is affected by stroke the distribution of coherence changes with stroke severity, demonstrating that low within-limb coherence is more likely with increasing motor deficit.

FIG. 27 displays the performance of an algorithm for detection of stroke when evaluated for a test data set using signal complexity, a single measure. The analysis was performed for data acquired in the continuous mode. Here, the dark shading indicates that a stroke was detected and light shading indicates that a stroke was not detected. The data from normal subjects (“N”) was correctly identified most of the time and stroke detections were not made for these data. The TNR, estimated from the normal subject data, was 0.998. Or there was one false positive every 21 days. For subjects with NIHSS (motor component) 1-4, the TPR was 0.50, 0.96, 0.99, and 0.96, respectively. A stroke with NIHSS=1, is a mild stroke which does not necessitate treatment with tPA. For NIHSS 2-4, that is, for moderate to severe strokes which need timely intervention and are our target, the TPR was 0.97.

FIG. 28, a pie chart 2800, shows an example of multinomial classification for data from normal subjects and from stroke subjects, with the stroke data being labelled with five degrees of stroke, from quasi-normal to severe. The analysis was performed for data acquired in the continuous mode. The classification was obtained through the joint use of several computed features. The algorithm is perfectly accurate, with no false positives for non-stroke (i.e., normal) subjects and subjects with motor deficit difference 0 (quasi-normal), 2 (moderate) and 4 (severe). Overall, there were false positive classification decisions in only 4.1% of the subjects.

FIG. 29 illustrates a bar graph showing the accuracy of detecting a motor deficit difference (asymmetry) between the affected and unaffected arm. The analysis was performed for data acquired in the continuous mode. Motor deficit of 0 indicates no motor drift in both arms while motor deficit 4 indicates extreme difference in motor drift (usually one arm with no ability to move while the other arm has no drift). Here the metric shown represents the accuracy of classifying the level of each motor deficit vs all other labels. For example, the value shown for motor deficit 1 represents the accuracy of detecting a motor deficit score of 1 with respect to all other labels.

FIG. 30 illustrates an average receiver operating characteristic (ROC) curve for multi-binary model predictions. The results show a classification between the average ROC of normal/mild (motor deficit difference of 0 and 1) vs the rest and average ROC of moderate to severe (motor deficit difference of 2, 3, and 4) vs the rest for a test cohort. The analysis was performed for data acquired in the continuous mode.

FIG. 31 shows an example of acceleration data reflecting a normal subject's upper limb movements when performing a prescribed task. Here both limbs are free of motor deficit. Similar behavior is observed for both limbs, resulting in qualitatively identical acceleration graphs.

FIG. 32 shows an example of acceleration data reflecting a stroke subject's upper limb movements when performing a prescribed task. Here, one of the limbs has a motor deficit. The presence of a motor deficit manifests with a difference in the behaviors of the limbs and results in qualitatively distinct acceleration graphs.

FIG. 33 shows examples of quantification of acceleration data using coherence analysis (left) and statistical analysis (right) when both user limbs are free of motor deficit. The analysis was performed for data acquired during a task. The maximum coherence over all frequencies is close to the theoretical maximum 1.0, and the frequency at which the maximum coherence is observed can be readily identified (left). Also, the standard deviations of both acceleration signals are considerable and similar (right).

FIG. 34 shows examples of quantification of the acceleration data using coherence analysis (left) and statistical analysis (right) when one of the limbs has a motor deficit. The analysis was performed for data acquired during a task. The presence of a motor deficit results in the cross-limb coherence having values close to 0.0, with the frequency of motion being poorly identifiable (left). Also, the standard deviation of the acceleration recorded on the affected side of the body (left) is less than that on the healthy side (right).

FIG. 35 illustrates the accuracy of the developed models, for the analysis of data acquired during the performance of tasks, for a multinomial classification of subjects with different levels of severity of stroke. A combination of three tasks out of the total battery of 38 tasks was used. The numbers on the main diagonal of the confusion matrix are the percentage of true classifications, while the off-diagonal entries are the corresponding percentages of misclassification. We note that the percentage of misclassifications rapidly decays with distance from the diagonal (i.e., from the true classification). This result provides strong evidence for the accurate identification of stroke severity from data recorded during tasks.

FIG. 36 illustrates the accuracy of the developed models for a multinomial classification of stroke severity as a function of the number of tasks. For the majority of the developed models, the use of one or two tasks results in accuracy which has room for improvement. At the same time, an increase in the number of tasks beyond three tasks leads to a slight loss of accuracy. This suggests that a 3-task combination is optimal for the accurate identification of the severity of stroke.

FIG. 37 illustrates the accuracy of the algorithms developed for the classification of activities of daily living. The analysis was performed for data acquired in the task-based mode. The figures display the ROC curve and the area under the curve (AUC) which result from the activities of daily living classification algorithm for data from both stroke subjects and healthy controls (left) and for data from healthy controls only (right).

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

Referring to the various figures, the present invention provides a real-time automated system and method to diagnose and/or detect stroke and engage the patient, care-takers, emergency medical system and stroke neurologists in the management of this condition. The present system and method for diagnosis, detection, and notification regarding the onset of a stroke (hereinafter “system 10”), employs body worn sensors 12 and a cloud-based patient specific analysis to dramatically shorten the time to definitive therapy for patients with ischemic stroke.

The system 10 continuously measures natural limb activity and conveys these measurements to a cloud based real-time data processing system 14, which both identifies patient specific alert conditions and determines solutions for acting upon the needs of the specific patient (or user, or subject) 100. The system 10 further provides for notification of potential stroke syndromes in real-time and activation of acute stroke protocols. The system 10 also provides the ability to interact with the patient, to assess limb activity, physiology, and cognitive elements (such as audio and visual cues). It is appreciated that the present system 10 is particularly suited for those individuals predisposed to the occurrence of a stroke, for example, individuals who have previously suffered a stroke, individuals with high blood pressure, individuals with a family history of strokes, etc.

As will be explained below in great detail, the system 10 relies upon three components:
1) a body worn sensor(s) 12 that is preferably worn on the limb(s) of a patient;
2) a patient worn or proximal data transmission device 16; and
3) a cloud-based data processing system 14 acquiring information (in the form of data) generated by the body worn sensor 12 and transmitted by the data transmission device 16, analyzing the acquired information, identifying the potential onset of a stroke, and notifying the patient, a patient's caretaker(s), an emergency medical system(s) and/or stroke neurologist(s).

As will be explained below in greater detail, the data processing system 14 performs the step of identifying the potential onset of a stroke by first establishing a patient specific limb activity signature through the aggregation of continuously sampled data acquired over minutes, hours, days, weeks and months, and the application of analytical methods as discussed below in greater detail. The system 10 facilitates interaction with the patient and the patient's caretaker(s). Further, the system 10 initiates the steps necessary for the transfer of the patient for rapid evaluation.

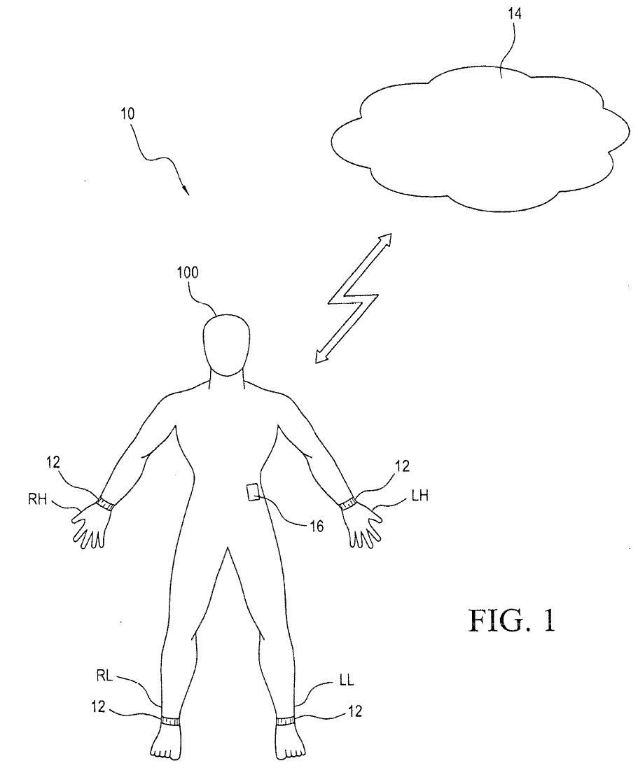

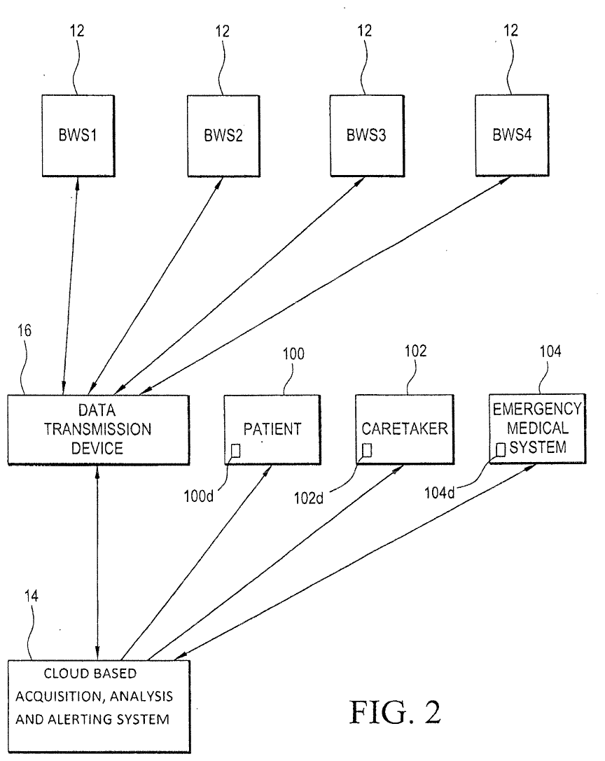

Referring to FIGS. 1 and 2, and as will be explained in greater detail below, the flow of information begins with sensor readings acquired by one or more body worn sensors 12 (referred to as BWS1-BWS4 in FIG. 2). The body worn sensors 12, for example, may be composed of four limb worn sensors 12, as well as other body worn sensors that may be worn on other parts of the body of the patient 100. The data collected by the body worn sensors 12 is relayed to the patient worn or nearby data transmission device 16 (for example, a smartphone 35 with a computer software application or “app” 36 operating in accordance with the present invention). A time-stamp is added to this data stream by the data transmission device 16 and the data from the multiple body worn sensors 12 is compiled into a single data stream. The data is then relayed by the data transmission device 16 to the cloud-based data processing system 14, in particular, the acquisition system 40 of the cloud-based data processing system 14.

Cellular and/or Internet communication networks are preferably used to transfer the data from the data transmission device 16 to the cloud-based data processing system 14. With this in mind, it is appreciated there are two general ways in which the problem of early detection of stroke may be approached. In accordance with one approach, the environment in which the patient resides is instrumented (that is, the majority of sensors picking up patient activity and functional data transmission components are positioned within the environment in which the patient resides; for example, sensors in the floors, and on the walls, and through cameras and microphones, wherein these sensors would continuously monitor the patient to determine the subject state and level of activity), and continuous readings are made through sensors embedded within the environment, which are then transmitted to the cloud based data processing system 14 for evaluation. In accordance with a second approach, the patient 100 is instrumented. That is, functional components allowing for data transfer in accordance with the present invention are integrated into the body worn sensors 12 and the data transmission device 16.

In accordance with a preferred embodiment, and as will be appreciated based upon the following disclosure, the system 10 employs an approach wherein the patient 100 is instrumented; that is, the system 10 provides a solution which is centered around the patient 100, and does not depend on the environment. In this regard, cellular data transmission is the preferred mechanism for data transfer in accordance with the present invention. However, and if the environment does support secure Wi-Fi (a trusted network is detected), then the data transmission device 16 switches to Wi-Fi when and where it is supported. For the patient 100, the ability to switch to a trusted Wi-Fi network will save data on a cellular plan. It also reduces power consumption and heat generation and increases battery life because Wi-Fi transmission is over a few meters and cellular transmission is over a few km.

In general, the present system 10 is open to both solution strategies; one where the patient 100 is instrumented and one where the environment is instrumented. The solution in which the environment is instrumented will become increasingly more important as nursing homes, rehabilitation facilities, and homes become more instrumented. It is further appreciated that as the system 10 develops, an increasing number of sensors may be available within the environment, and it will be desirable to use data from those sensors in addition to the data from the body worn sensors 12 on the patient. Optionally it is appreciated that the data could also be transmitted directly from body worn 12 and environmental sensors to the cloud 14 without the need of a data transmission device 16. That is, the solution could preclude the data transmission device if the capability to communicate with the cloud is included in the body worn sensors.

The acquisition system 40 of the cloud based data processing system 14 adds an additional time-stamp, and conditions the data with nulls if there are missing data samples. With this in mind, it should be appreciated that the patient worn data transmission device 16 is a hub between the body worn sensors 12 and cloud based data processing system 14. The patient worn data transmission device 16 always knows which data it has received and which data it has not received from the body worn sensors 12. When data is not received from a particular body worn sensor 12, the patient worn data transmission device 16 indicates this to the cloud based data processing system 14.

The patient worn data transmission device 16 always conveys to the cloud based data processing system 14 how many body worn sensors 12 it is in contact with. For example, patient worn data transmission device 16 is expected to be in contact with body worn sensors 12 worn on the LH (left hand or arm), RH (right hand or arm), LL (left leg), and RL (right leg) of the patient 100, and periodically (currently at the start of each second) informs the cloud based data processing system 14 know it is in contact with these four body worn sensors 12. That is, at the start of each second the patient worn data transmission device 16 transmits to the cloud based data processing system 14 the codes, for example, LH, RH, LL, RL, to indicate that it is receiving data, respectively, from those four body worn sensors 12.

If a few data samples are not received from a body worn sensor 12, the patient worn data transmission device 16 marks this with a code value in lieu of the data values to indicate those data were missing. If the data stream from a body worn sensor 12 stops (e.g., currently if no data is received for >1 sec), then the patient worn data transmission device 16 indicates this to the cloud based data processing system 14 by updating which body worn sensor(s) 12 it is in contact with. That is, for example, for the instance where the RL body worn sensor has stopped communicating, at the top of the next second it indicates that it is in contact with LH, RH and LL body worn sensors 12, and not the RL body worn sensor 12.

The data is then analyzed on the cloud based data processing system 14 and alerts are sent to the patient 100, the patient's caretaker(s) 102 and the emergency medical system(s) 104 as required. Alerts may be sent via text message, email, or other mechanisms to communication devices 100d, 102d, 104d of the patient 100, the patient's caretaker(s) 102 or the emergency medical system(s) 104 in a manner known to those skilled in the art.

### I. BODY-WORN SENSOR

The first part of the system 10 is the body worn sensor(s) 12 shaped and dimensioned to be worn on limb(s) of a patient 100. In accordance with a preferred embodiment, it is contemplated either two or four limb worn sensors 12 are used, wherein the body worn sensors 12 are respectively worn on the left hand or arm (LH), the right hand or arm (RH), the left leg (LL), and the right leg (RL) of a patient 100. If only two sensors are used, they will preferably be worn on the left hand or arm (LH) and right hand or arm (RH). If four sensors are used, they will be worn on all four limbs.

As will be explained below in greater detail, the four limb worn sensors 12 each have a 3-axis, 6-axis, or 9-axis accelerometer and a temperature (T) sensor. Additional sensors may be used on the body to capture additional information. Further, one or more of the four limb worn sensors 12 may also measure galvanic skin response (GSR) and heart rate (HR). The body worn sensors 12 are preferably as unobtrusive as possible. As such, it is contemplated the body worn sensors 12 may be integrated within ordinary objects which are worn on the body such as, but not limited to, clothing, cufflinks, buttons, shoe or belt buckle, ring, bracelet, shoelaces, eyeglass frame and as a patch on the body, including on the chest.

As will be appreciated based upon the following disclosure, detection events indicative of the onset of a stroke are triggered by a change in the signals from expectation. The primary detection signal is based on anomalous asymmetry in limb activity. The present system 10, however, uses additional sensors such as HR sensor 60, GSR sensor 64, pressure (P) sensor 26 and T sensor 66 to discover other (anomalous) changes which may occur in the peri-stroke period. If these contribute, directly or indirectly, to a detectable difference during the peri-stroke time from baseline, then they are incorporated into the detection process. Though a preferred current approach does not depend on it, all activity is categorized into recognizable states. For example, the system 10 evaluates if it is possible to detect when a person is standing, sitting, or lying down; OR when a person is awake or asleep; OR when a person is indoors or outdoors; in a car (or bus, train), or not; OR when a person is engaged in common tasks such as walking, eating, exercising, getting dressed/undressed. The purpose of this is to improve upon the ability to detect anomalies. The better the system 10 is able to recognize normal states, the better will be the ability to detect anomalies.

Each of the body worn sensors 12 independently and continuously samples limb activity and adds a timestamp to it. The signals generated by the body worn sensors 12 are processed to minimize data size, without loss of information, and the signals with the timestamp are then wirelessly communicated to the data transmission device 16.

In accordance with a preferred embodiment, data size is minimized primarily to minimize battery power consumption, transmission cost, and heat generated. Conversely, it is desired to transmit data as close to the full raw sensor data as possible because it is desired that such data should be accessible to analysis programs which run on the cloud-based data processing system 14. It is appreciated that the data stream for this application is not excessive, and the data bandwidth is managed, for the reasons indicated above, in the following manner:
1) Data is sampled at a low frequency. Patients 100 are often not very active, so the sampling frequency can be reduced to a low rate. In accordance with an embodiment, the accelerometers are sampled at 40 Hz, and it may be possible to sample at 25 Hz. In accordance with another embodiment, the accelerometers are sampled at 25 Hz, and it may be possible to sample at 50 Hz or 12.5 Hz. Modalities such as GSR and T, can be sampled less frequently (for example, they can be sampled once every sec, or less frequently).
2) Data samples are transmitted as raw A/D values, that currently are 2-byte integers, although it is appreciated more than 2-byte integers may be used as the resolution of the sensors improves.

In accordance with a preferred embodiment, all data samples are transmitted. As the characteristics of specific patients are understood through implementation of the present invention, the data transmission device 16 filters out immaterial data so as to limit the data that is ultimately transmitted to the cloud-based data processing system 14. In attempting to optimize battery consumption, heat generation, and data throughput, transmission in bursts may be employed in accordance with the present invention.

The body worn sensors 12 include similar characteristics and a single representative body worn sensor 12 is described below. With this in mind, the system 10 may include various arrays of body worn sensors; for example, it may be desirable to provide limb worn sensors to pick up limb activity, and then additional sensors on one or two limbs, or another location such as the chest, or ear, where HR and GSR are measured and detected. The data transmission device 16 includes a microphone 16a and a camera 16b, and is used to also pick up audio (the person speaking) and video/snap shots of the person or the person's face if necessary.

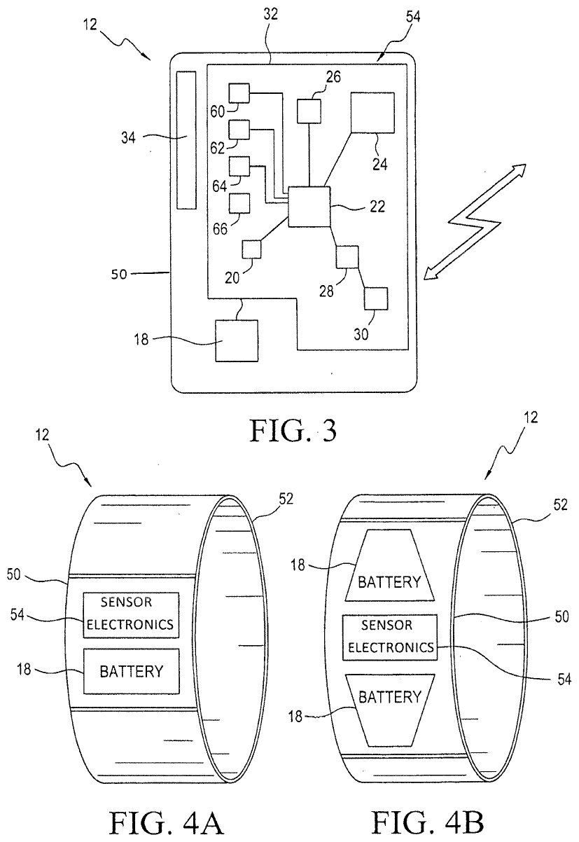

The body worn sensor 12 is constructed to continuously identify limb position in conjunction with the duration of limb movements, and the speed of limb movements (or otherwise referred to herein as “limb activity”). Referring to FIGS. 3, 4A and 4B, the body worn sensor 12 includes a housing 50 for electronics associated therewith and a strap 52 for wrapping about the limb of the patient 100. In particular, sensor electronics 54 and a power source 18 (in the form of a battery, wherein the first embodiment shown in FIG. 4A includes a single battery 18 and the second embodiment shown in FIG. 4B includes dual batteries (or more than one battery) 18) embedded within the housing 50. The sensor electronics 54 include a real time clock 20, a data processor 22 (for example, a microcontroller) controlling operation of the body worn sensor 12, a 3-, 6- or 9-axis motion tracking device 24, a pressure sensor 26 (optional), a temperature sensor 66, a signal processor 28 continuously processing signals generated by the motion tracking device 24, and a wireless communication module 30 (supporting, for example, Bluetooth Low Energy) continuously transmitting signals to the data transmission device 16. In selecting whether to utilize a pressure sensor, it should be appreciated that pressure sensor or difficult to incorporate into a system such as that disclosed herein and exhibits relatively high power consumption.

In addition to these elements, other sensor elements may be employed in conjunction with (or integrated with as is shown in FIG. 3 in accordance with a preferred embodiment) the body worn sensor 12. Such additional sensor elements may be worn on the limbs (in conjunction with the body worn sensors 12 described above, on the torso or on the ear. The additional sensors may include, but are not limited to, a heart rate (HR) monitor 60, a 1 or 2 lead EKG (electrocardiogram) 62 (which would necessarily be worn on the torso; and those skilled appreciate various other EKG systems may be used in accordance with the present invention), a galvanic skin response (GSR) monitor (an electrodermal activity or skin conductance monitor) 64, body temperature monitor (T) 66, a sweat analysis monitor, a hydration monitor, a muscle tautness monitor, a blood composition monitor (achieved through spectrometry), a sleep monitor, an audio monitor, a video monitor and/or other monitoring devices that might provide information useful in the detection of the onset of stroke and other acute central nervous system injuries.

More particularly, and in accordance with a preferred embodiment of the present invention, the data processor 22, motion tracking device 24, signal processor 28, wireless communication protocol (including an antenna) 30, and other sensing devices are integrated into a single printed circuit board 32. Preferred elements for the present body worn sensor 12 include a power source 18 in the form of a coin cell battery, a rechargeable battery, a flexible thin film battery or a thin film solid state battery. The motion tracking device 24 is preferably composed of the InvenSense MPU-9255 or similar device. The InvenSense MPU-9255 combines a 3-axis gyroscope, 3-axis accelerometer, 3-axis magnetometer, and a digital motion processor to produce an output signal indicating the position and orientation of a limb to which the body worn sensor 12 is attached. Further, the data processor 22 is preferably composed of a low power microcontroller unit and a wireless communication protocol 30, for example a Bluetooth Low Energy transceiver. Still further, the body worn sensor 12 includes a button and LEDs for user interaction. Additionally, it may include an interface display 34 having a touch screen allowing for various tap gestures used in the operation of the present body worn sensor 12. In accordance with a preferred embodiment, the communication between the body worn sensors 12 and patient worn data transmission device 16 is performed over Bluetooth Low Energy (BLE). The communication in accordance with a disclosed embodiment follows the BLE 4 GATT data protocol. In accordance with current embodiments, the communication follows the BLE 5 GATT data protocol. In the future it is anticipated this will change to newer releases of the BLE or other communication protocols that may become available. The data are transmitted from sensors to the data transmission device in a data stream of 16 bit (2 byte) words. In the patient worn data transmission device 16, the different sensor data streams are combined into one data stream, and a time-stamp is added. It is, however, appreciated that the different data streams may be combined in various ways depending upon specific implementation of the present invention.

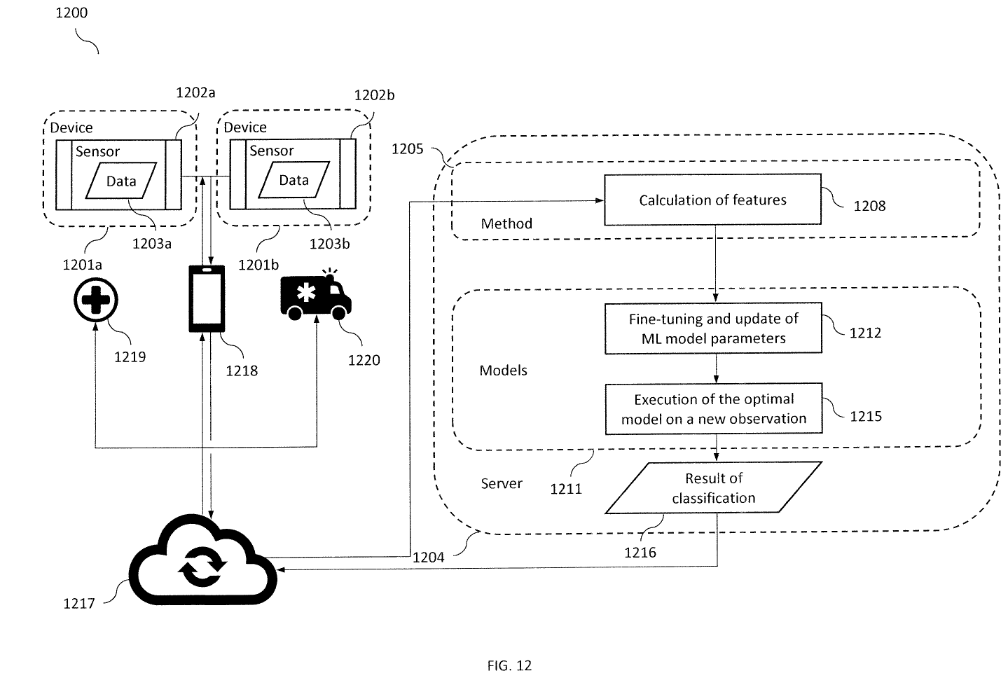

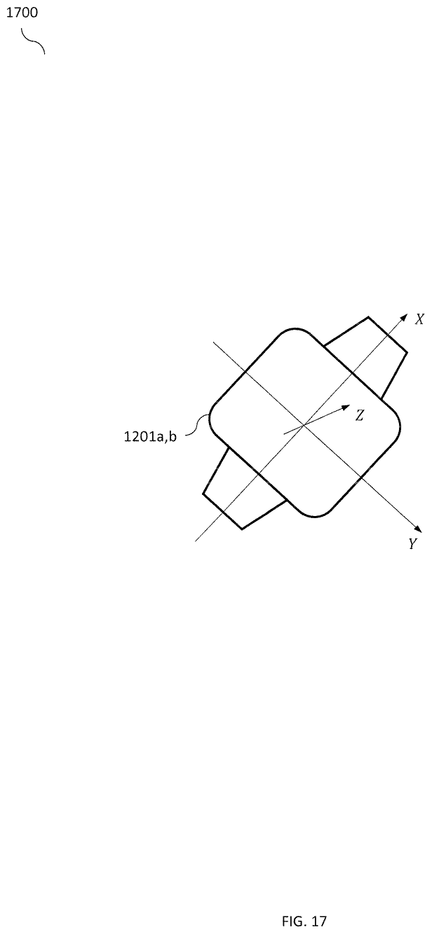

In accordance with a disclosed embodiment as discussed below with reference to FIG. 12, the wearable devices 1201a, 1201b with an example of a sensor-related coordinate frame XYZ are shown in FIG. 17 as a general schema 1700. While the sensors 1202a, 1202b of the wearable devices 1201a, 1201b collect the data 1203a, 1203b, these are recorded with respect to the coordinate frames of the sensors 1202a, 1202b which are continuously changing their orientation in space. For each component of the acceleration vector the data 1203a, 1203b is being recorded. In the implementation described here the sampling rate is at least 12.5 Hz. Although in FIG. 17 the sensor-related coordinate frame is illustrated to contain three axes, which implies recording a three-dimensional acceleration vector, the sensors 1202a, 1202b can also measure orientation in space, thus recording from one to three angular rotations. In some implementations the sensors 1202a, 1202b can also measure magnetic field around the device, thus recording, depending on the technical characteristics, the direction, strength, or relative change of the field.

As discussed below in detail, the firmware on the wearable devices 1201a, 1201b supports several features to maintain accurate continuous monitoring for extended periods of time extending to days, weeks, and months. The accuracy of a real-time clock on a wearable device depends on several factors and the clock on current wearable devices can drift and be either advanced or delayed by several seconds in a day. Since we use multiple devices 1201a, 1201b, a smartphone 1218, and a cloud backend 1204, which all have independent clocks, this can result in a lack of synchrony. In accordance with a disclosed embodiment, the smartphone clock is considered to be the master clock and constantly measures and corrects wearable device clock drift with respect to the smartphone clock. This solution maintains very close synchronization (within a few milliseconds) of the wearable device 1201a, 1201b and smartphone clocks indefinitely. In addition, the wearable devices 1201a, 1201b communicate with the smartphone 1218 over a radio link (e.g., Bluetooth low energy (BLE)). Given the constraints of a wearable form-factor on the antenna and radio power, BLE is susceptible to dropped links. This is not desirable for a solution which performs continuous monitoring in the background. This issue is addressed by constant monitoring of the communication link and seamless re-negotiation of it if the link is lost. This re-negotiation does not require user intervention and is handled in the background. In the event of a dropped BLE link, the data 1203a, 1203b is buffered on the wearable devices 1201a, 1201b, with data being transmitted to the smartphone 1218 and cloud 1204 in the background once the link is re-established. That is, the device firmware ensures no data is lost if the radio link is dropped.

### II. DATA TRANSMISSION DEVICE

The data transmission device 16 continuously receives data from the body worn sensor(s) 12, timestamps the sensor data, compiles the sensor data into a single synchronized data stream, and transmits the data to the cloud-based data processing system 14. It is appreciated that while the body worn sensor and the data transmission device are disclosed herein as separate and distinct components, these components could be combined into a single unit.

The data structure holds 1-sec of data as follows:
At the top of each second is a header with a timestamp and a set of codes to indicate which body worn sensors 12 (that is, the body worn sensors 12 of the LH, RH, LL, RL) and modalities are recorded.
Following this, are the data samples are recorded by the different body worn sensors 12. If sampling occurs at a rate of 50 Hz, then a set of data samples is received from each body worn sensor 12 every 20 msec. If sampling occurs at a rate of 25 Hz, then a set of data samples is received from each body worn sensor 12 every 40 msec.
Each set of these data samples has the following structure:

$$< x 1 > < y 1 > < z 1 > < x 2 > < y 2 > < z 2 > < x 3 > < y 3 > < z 3 >$$

Here:
<x1><y1><z1> are the three acceleration readings
<x2><y2><<2> are the three gyroscope readings
<x3><y3><z3> are the three magnetometer readings.
As indicated above, each of the data samples <x1> or <y1> etc. is a 2-byte word.
In addition to this, the accelerometer (not shown) within a patient worn data transmission device 16 (for example, the accelerometers that are conventionally included in smartphones 35) can also be sampled. This may be a 3-axis, 6-axis, or 9-axis accelerometer.
The body worn sensors 12 which are sampled less frequently, for example, GSR sensors 64 and T sensors 66, which are, for example, sampled only once per sec in accordance with a disclosed embodiment, are stored next.
Finally, a set of flags and data values indicate the state of the communication of this data to the cloud-based data processing system 14. This indicates the number of times an attempt has been made to transmit the data to the cloud and information on the communication used (cellular, Internet, network), whether the data were streamed live or from the storage on the wearable device.
Other modalities, e.g., audio via the microphone 16a, video via the camera 16b, and GPS are processed in separate data streams (as supported by the operating system of the patient worn data transmission device 16) and are collected when needed.
The battery state and strength of communication between the body worn sensors 12 and the patient worn data transmission device 16 is embedded within the data stream and transmitted to the cloud-based data processing system 14 periodically, for example, once a minute.
Once data have been successfully received from the body worn sensors 12 by the data transmission device 16, they are removed from the body worn sensors. Similarly, once data have been successfully received from the data transmission device 16 by the cloud-based data processing system 14, they are removed from the data transmission device.

In accordance with one embodiment, the data transmission device 16 provides a timestamp regarding the date and time the limb activity is received. While the time-stamp as discussed herein takes place in the data transmission device 16, it is appreciated the data is also time-stamped as it is developed in the body worn sensor 12 and when it is received by the cloud-based data processing system 14.

In accordance with a preferred embodiment, the data transmission device 16 is preferably a smartphone 35 employing an “App” 36 to achieve data transmission in accordance with the present invention, as well as electronics 37 commonly employed in a smartphone 35. In accordance with a preferred embodiment, the smartphone operates under Google's ANDROID® operating system or Apple's iOS operating system, or other operating system that may be developed in the future. It is also appreciated tablets or other similar electronic devices may be used instead of smartphones. In the event of a notification regarding the onset of a stroke or notifications regarding other elements of the present system 10, the smartphone 35 is also used as the contact mechanism for communicating with the patient. Although a smartphone is disclosed for use in accordance with the present invention, it is appreciated other data transmission devices may be employed without departing from the spirit of the present invention.

### III. CLOUD-BASED DATA PROCESSING SYSTEM FOR ACQUISITION, ANALYSIS AND ALERTING

The third part of the system 10 is preferably a patient data privacy compliant (for example, HIPAA (Health Insurance Portability and Accountability Act compliant)) cloud-based data processing system 14 acquiring and processing the data streamed from each patient 100. The cloud-based data processing system 14 compiles data, and detects, using a patient specific database and powerful time-series analysis methods, patient specific limb activity signatures representative of change due to stroke. It is appreciated that the time-series analysis method employed in accordance with the present invention could be supplemented by adding any number of direct or indirect measures where such measures are determined to improve the performance of the time-series analysis method. While limb activity data is the primary modality employed in the analysis method, other sensor modalities, for example, audio, video, heart rate, temperature, etc., may be employed in identifying the onset of a stroke. Based upon patient specific limb activity signatures, the cloud-based data processing system 14 identifies the likely onset of a stroke, that is, an alert condition, and initiates protocols for subsequent interaction with the patient, or the patient's caretaker(s). The interaction with the patient 100 may include acquisition of audio or video/snap-shots. The audio is analyzed to determine changes in speech. The video/snap-shots are analyzed to determine changes in facial muscles or posture indicative of stroke. The cloud-based data processing system 14, in conjunction with the identification of the likely onset of a stroke (or other acute central nervous system injuries), also initiates related treatment protocols, for example, activation of the emergency medical response system, the transport of the patient to the nearest Neurocritical Care Unit or emergency room for rapid evaluation and treatment.

The cloud-based data processing system 14 is composed of three parts: an acquisition system 40; an analysis system 44; and a patient management system 46. Additionally, the cloud-based system includes components to help monitor the status of the cloud-based system. This includes automated and semi-automated tests of data ingestion, analysis, storage, notification, and documentation.

### A. Acquisition System

The acquisition system 40 of the cloud-based data processing system 14 compiles all the streaming data generated by the body worn sensor(s) 12 and transmitted by the data transmission device 16. For each patient 100 being monitored in accordance with the present system 10, the received data is stored in a patient specific database 42. In conjunction with the compilation of the received data, the acquisition system 40 also determines connectivity of the body worn sensor(s) 12 and the quality of acquired data. In accordance with a preferred embodiment, the compilation of the received data and the determination of sensor connectivity are achieved by assessing the time-stamped data sent by the data transmission device 16, identifying gaps in the data, and formatting the data for further processing. For example, the 9-axis motion tracking device 24 of the body worn sensor 12 continuously samples limb activity. The 9-axis motion tracking device 24 reads out three 16-bit accelerometer outputs (to measure acceleration along the X-, Y-, and Z-axes), three 16-bit gyroscope outputs (to detect rotation about the X-, Y-, and Z-axes), and three 16-bit magnetometer outputs (for detecting terrestrial magnetism in the X-, Y-, and Z-axes). Limb activity is determined from these nine measurements. The output of the 9-axis motion tracking device 24 is nine 16-bit words (that is 18 bytes) for each acquired sample. In accordance with an embodiment, sampling is at 40 Hz; that is, 40×9×18 bytes per second are generated, although other sampling rates could be used in accordance with various implementations. This data is streamed from each body worn sensor 12 to the data transmission device 16. It is appreciated that the sampling frequency is adjusted to optimize power consumption and capture of salient aspect of limb activity. It is further appreciated that other sensors may be added within the body worn sensors, and other sensors which are placed on the torso (for example, EKG sensors 62) or elsewhere on the body may be incorporated into the present system 10.

This data, with flags and time-stamps, is transmitted to the cloud-based data processing system 14 where it is processed for further analysis in accordance with the present invention. It should, however, be appreciated that the movement analysis is performed in the second step (i.e., in the analysis system 44 as described below). The process performed by the acquisition system 40 is straightforward in that the acquisition system 40 time-stamps the received data, and merges it with the previously acquired data.

With the foregoing in mind, operation of the acquisition system 40 may require an evaluation of continuity, and introduction of blanks (or NaNs) as filler data. In addition, the acquisition system 40 flags a patient's data stream for being discontinuous if there are many gaps in the stream. If the data are flagged in this manner, then that information is relayed back to the transmission device and the wearable device to help improve data connectivity, for example by increasing the radio power of a wearable device which has poor connectivity.

As indicated above, detection events for the identification of the onset of a stroke are triggered by the detection of a change in the signals. By this it is meant that the system 10 has an expectation of the signal characteristics for a given patient and deviations from this expectation are of interest as they may represent an event of interest, in particular, the onset of a stroke. All anomalies in the data stream are identified and analyzed. Any change in the data triggers a set of responses geared to understand why the change has occurred. This set of evaluations includes ruling out technical issues as a cause for the observed change. For example, it could be that the system 10 could suffer a loss of communication between the body worn sensors 12 and the smartphone 35, or between the smartphone 35 and the cloud based data processing system 14, or from the cloud based data processing system 14 to the patient 100; it could also be the case that this loss of one or more links could be due to technical reasons, and may have no implication on the health of the patient 100; or, it could be that that loss is reflective of an important change in patient state, beyond the technical issue, for example, a fall could jar a wrist worn sensor 12 loose, in which case it would transmit non-physiological data and there would be a change in the temperature measurements from body temperature to ambient temperature, or break it, in which case connection with the sensor 12 would be lost. Ultimately, the cloud-based data processing: system 14 builds a collection of analyzed, high quality data for each patient 100 and assesses all changes in the acquired signals.

### B. Analysis System

The data acquired is continuously analyzed using the analysis system 44. The system 10 tracks an ensemble of diffusion geometry measurements and other time-series measures of signal magnitude, variability, complexity, and interrelation determined from the measurements of the body worn sensor 12. The system 10 continuously matches ensembles of measurements from limb movement to detect disruption of symmetry, and track as well ensembles of measurements built from HR, GSR and T and other sensor modalities to detect anomalies. In accordance with the present invention system 10, the analysis system 44 employs the data processing methods developed by Professor Ronald Coifman of Yale University. These methods are described in Coifman, R. R. and S. Lafon, Diffusion maps. Applied and Computational Harmonic Analysis, 2006. 21 (1): p. 5-30, Coifman, R. R., S. Lafon, A. B. Lee, M. Maggioni, B. Nadler, F. Warner, and S. W. Zucker, Geometric diffusions as a tool for harmonic analysis and structure definition of data: Diffusion maps. Proceedings of the National Academy of Sciences of the United States of America, 2005. 102 (21), and Talmon, R., S. Mallat, H. Zaveri, and R. R. Coifman, Manifold Learning for Latent Variable Inference in Dynamical Systems. IEEE Transactions in Signal Processing, 2015, all of which are incorporated herein by reference.

Employing the diffusion mapping processes developed by Professor Coifman, the analysis system 44 continuously processes limb activity and sensor data, and determines, in comparison to a previously determined patient specific limb activity signature if the current limb movements of the patient 100 are within expected parameters. The activity of major muscle groups and the nervous system control of these muscle groups are quantified through the present system 10. This is accomplished by quantifying the geometry of the dynamics of the motion of limbs of a patient. Multi-dimensional representations of limb activity are built using diffusion maps, and the differences between a pre-established multi-dimensional representation of limb activity (that is, the patient specific limb activity signature) and a current multi-dimensional representation of limb activity are evaluated to identify changes from baseline (that is, the patient specific limb activity signature). If the limb activity is not within expected parameters (that is, the current activity is not consistent with the patient specific limb activity signature) then an alert is generated. Diffusion geometry enables precise quantification of distance between data clouds (that is, distance between ensembles of measurements characteristic of a patient). The system 10 has precise statistical measures of the differences between ensembles of measurements, these measures are designed to test the hypothesis that a patient 100 conforms to their own normal model, and where measurements do not conform to the patient's normal model the anomaly is documented and appropriate action is taken. In accordance with a preferred embodiment, the alert is issued within a time-window of 10-30 minutes. However, it is appreciated that it is possible that in the case of the introduction of subtle deficits in limb activity that the time to alert may be longer, as it may take longer for the analysis system 44 to detect the deficit in limb activity. It is also possible that in the case of sedentary patients that the analysis system 44 will take longer to raise an alert; once again, long determination times will be a result of the difficulties in definitively determining when an alert condition has been crossed.

More particularly, as to identifying anomalies indicative of the onset of a stroke, that is, the analysis leading to a determination of a patient specific stroke condition, the system 10 continuously quantifies limb activity, creates patient specific limb activity signatures, and compares real-time limb activity to a patient specific limb activity signature for the identification of anomalies that may be indicative of the onset of a stroke or other acute central nervous system injuries. Where anomalies are detected, alert conditions are identified and (as is described below with regard to the patient management system 46) alerts are initiated and action is taken.

In accordance with a disclosed embodiment, and as discussed above, the analysis system 44 develops limb activity signatures which are specific for each patient 100. These patient specific limb activity signatures are continuously compared against current patient behavior. In the event that a patient 100 has a deficit in limb activity introduced by a stroke, the previously established patient specific limb activity signatures can also be used to quantify the degree of success in restoring function during rehabilitation.

As briefly described above, this analysis relies upon analytical methods of Prof. Coifman for the analysis of sensor signals using diffusion maps. Diffusion maps are a dimensionality reduction and feature extraction algorithm. Briefly, the Eigen functions of a Markov matrix defining a random walk on the data are used to obtain new descriptions of data sets via a family of mappings that it is termed “diffusion maps.” These mappings embed the data points into a Euclidean space in which the usual distance describes the relationship between pairs of points in terms of their connectivity. This defines a useful distance between points in the data set that is termed “diffusion distance.” The approach employed by Coifman generalizes the classical Newtonian paradigm in which local infinitesimal rules of transition of a system lead to global macroscopic descriptions by integration. Different geometric representations of the data set are obtained by iterating the Markov matrix of transition, or equivalently, by running the random walk forward, and the diffusion maps are precisely the tools that allow the spectral properties of the diffusion process to be related to the geometry of the data set. In particular, one does not obtain one representation of the geometry for the set, but a multiscale family of geometric representations corresponding to descriptions at different scales.

As such, and in accordance with the present invention, data generated based upon patient limb activity is applied to create a multi-dimensional diffusional map as taught by Coifman. When this multi-dimensional diffusion map is created using data during normal, healthy movement, the multi-dimensional diffusion map defines a patient specific limb activity signature. This patient specific limb activity signature is then continuously compared with similar diffusion maps based upon real-time limb activity for the identification of anomalies indicative of the onset of a stroke. Anomalies are detected when the diffusion map based upon real-time limb activity deviates from the patient specific limb activity signature by a pre-established extent.

The ability to apply the diffusion mapping methodology to the present system 10 based upon identified anomalies in limb activity has been proven by collecting data from patients and normal volunteers, including using it to differentiate between runners based on the movements of their limbs.

In particular, two experiments were performed. The first experiment was performed on 11 individuals. These 11 individuals consisted of 6 normal subjects (5 female, age range 29-51 years, all right hand dominant), and 5 patients who had recently suffered a right hemisphere stroke (3 female, age range 47-86, 3 right and 2 left hand dominant). The NIH Stroke Scale/Score (NIHSS) was evaluated for the 5 patients before the study and was 11, 9, 1, 5, and 2. The left and right arm strength was evaluated and was 0/5, 0/5, 4/5, 4/5, and 4/5 for the left arm and 4/5, 5/5, 5/5, 5/5, and 5/5 for the right arm, respectively, (the stroke patients all had left side weakness). The 11 individuals wore a 3-axis accelerometer both on the left arm and the right arm. While wearing the sensors, participants were asked to perform a neurological exam, consisting of specific motions, for the purposes of obtaining standardized data. These motions followed elements of the neurological exam, with attention to motor strength, movement in planes, and gait. Also included were tasks such as lifting your arms up for 10 seconds, simulate washing your hands in the sink, simulate drying your hands with a towel, and flipping your palms up and down 5 times.

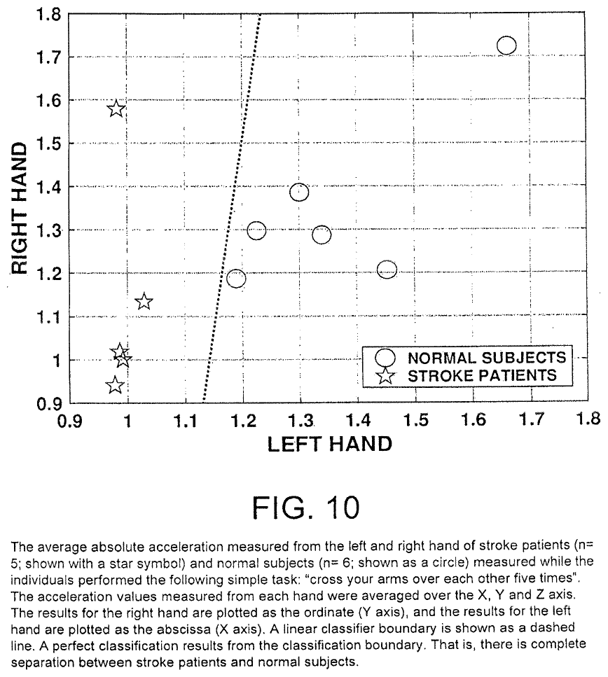

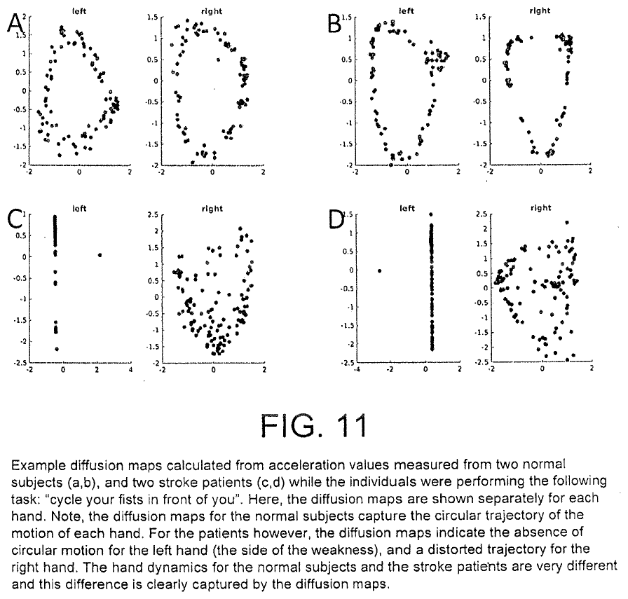

Acceleration data was collected during the performance of the tasks. The accelerations in the X, Y and Z axes for each arm was measured. The acceleration data were sampled at 25 Hz. The data were evaluated with different time-series analysis measures. Two sets of results are shown here to demonstrate the separation of the normal subjects and the stroke patients. First, in FIG. 10, the average absolute acceleration for the left arm and right arm for each individual tested is presented. The average absolute acceleration was first averaged separately for each of the three axes studied, and then an average was calculated for the three axes. The data used here were collected when the individuals were performing a simple task “cross your arms over each other five times.” The average absolute acceleration values for the 6 normal subjects and 5 stroke patients are shown in FIG. 10. A linear classification boundary was estimated and is displayed. The linear classification boundary indicates that there is a complete separation between the data from the stroke patients and normal subjects. In FIG. 11 the diffusion maps analysis of acceleration data from two normal subjects and two stroke patients while they performed a second task “cycle your fists in front of you” is presented. The diffusion maps demonstrate a clear capture of the dynamics of the arm movement, and the difference between the stroke patients and the normal subjects. Both a simple measure of magnitude, the average absolute acceleration, and the diffusion maps have been included to underscore the following. It is possible that a number of time-series measures can be used to distinguish between patients and normal subjects. However, to create limb specific signatures a more powerful methodology is needed to capture the dynamics of limb activity. The first method employed removes all information on the dynamics of limb activity, to obtain the average absolute acceleration value. This is sufficient to achieve a separation between patients and normal subjects, and thus is of value. The second method, diffusion maps, however, is a more powerful method which allows capture and quantification of the rich dynamics of these data and thus can further extend the analysis which is performed by the simple time-series measures. The ability of the diffusion maps algorithm to create limb specific signatures is illustrated through the second experiment described below.

In the second experiment, acceleration and rotation data was gathered from the wrist and ankles of thirteen runners at speeds 5 and 7.5 on a treadmill, and this data was used in conjunction with the diffusion maps feature extraction technique to identify unique characteristics between runners. Upon computing the diffusion map embedding for pairs of motion datasets, similarities between runners were computed by comparing their ergodic measures.

Thirteen volunteers (eight males, five females) between the ages of 18-22 were asked to run on a treadmill and produce motion data. The same treadmill was used for each trial for sake of pace consistency. Devices running a motion measurement app were strapped to each runner's right ankle and wrist. The workout is described below:
1 minute warmup, running at a speed of 6 mph (mile per hour) (no data collection)
30 second rest.
1 minute workout, running at a speed of 5 mph (data collected)
30 second rest.
1 minute workout, running at a speed of 7.5 mph (data collected)

Motion data samples were taken at 100 Hz, where each sample consisted of a 12-dimensional vector (6 values per ankle and wrist). Each workout consisted of two trials, one at a speed of 5 mph and one at a speed of 7.5 mph. A trial was defined to be the time series of 12-dimensional vectors at a given speed. Among the thirteen runners, five performed more than one workout, and the remaining eight performed only one workout, resulting in 40 total trials. Each trial generated a single dataset, for a total of 40 datasets.

Diffusion maps were created and used to reduce the high dimensional datasets obtained from individual runners and attempt to extract relevant features to distinguish between them. From these similarities between motion datasets generated from the same person were detected. A novel algorithm was thereafter generated that combines diffusion . . . maps for dimensionality reduction of a dataset and uses a generalization of the earth mover's distance as a similarity metric to compare datasets. Generally, this algorithm considers the various diffusion maps by applying eigenfunctions to the data and creating a broad representation diffusion map. With the plurality of diffusion maps generated in this manner, overlaps are identified and correlations are determined. For a given runner, the algorithm successfully found correlations between motion data at a speed of 5 mph with motion data at a speed of 7.5 mph for the same runner, and correlations also held for a given runner at the same speed over multiple workouts.

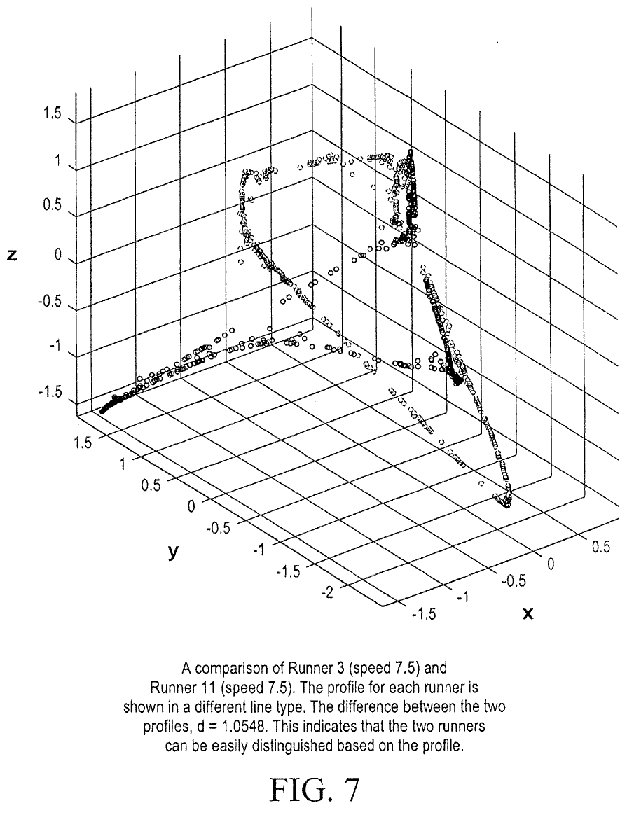

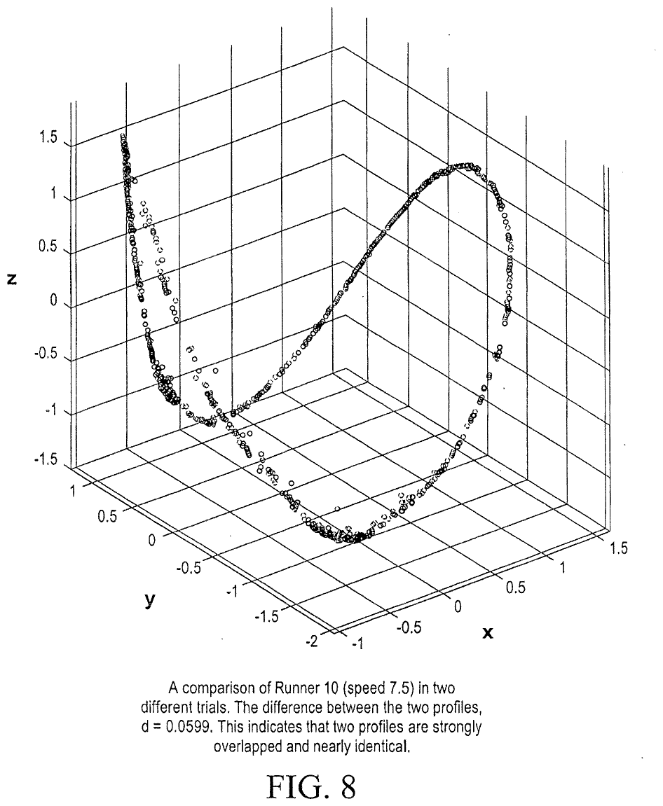

After applying the diffusion map algorithm to a pair of trial datasets, it was possible to visualize comparisons between trials by plotting the three most significant embedding vectors in MATLAB and coloring points in the embedding based on which original dataset they correspond to. Obvious differences between the trajectories of different runners, and trajectories produced by the same runner were observed by way of this technique. In FIG. 7, it is evident that distinct Runners 3 and 11 have vastly different running patterns, based on both the visual disparity in the scatter plot as well as the large distance between their ergodic measures, d=1.0548. On the other hand, FIG. 8 depicts two trajectories produced by the same runner at a speed of 7.5 mph that are much more similar both in appearance as well as the small distance between them, d=0.0599. Note that between trials, Runner 10 was given a period of rest and had the motion measurement devices re-strapped between trials, yet still exhibited high correlation across runs.

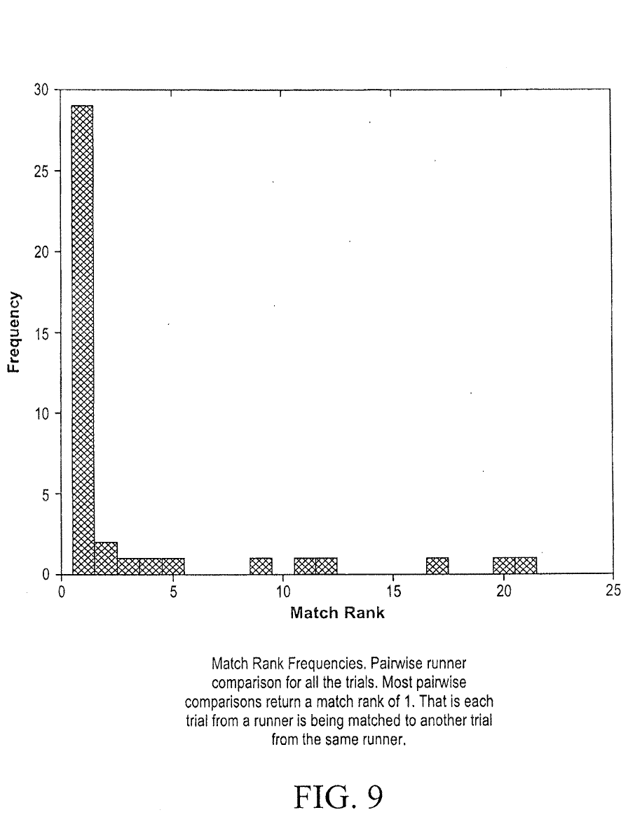

The most striking result of the study was that an individual runner's motion data at a speed of 5 mph is correlated with the same runner's motion data at a speed of 7.5 mph. Most of the runners ran only one workout, producing one dataset at a speed of 5 mph and one at a speed of 7.5 mph, and in the analysis, all datasets from both speeds were combined. A MatchRank measure was created to compare the similarity of the different trials. When MatchRank=1, the data from a patient is matched to data from the same patient in a pairwise comparison. Most pairwise comparisons return a MatchRank value of 1. That is each trial from a runner is being matched to another trial from the same runner. Frequency charts were created of MatchRanks producing compelling results displayed in FIG. 9. This method was able to produce a MatchRank of 1 in 29 out of 40 trials, for a success rate of 72.5%. Hence, many of the MatchRank values shown in FIG. 9 are 1 as result of a runner's speed at the 5 mph trial being matched with the speed at the 7.5 mph trial, indicating some underlying “running style” that is unique to an individual runner, even across varying speeds.

As with any experiment involving physical measurements, it is appreciated that inaccuracies in the data collection process may lead to error. Most notably, it was impossible to standardize device orientation across trials because the devices were strapped on by hand. Second, the fact that runners had smartphones strapped to their limbs also probably had a disruptive effect on their natural running patterns. Lastly, the motion sensing hardware was inconsistent as both an iphone 5c and iphone 4s were used to collect motion data. However, because of the minor physical differences between the two smartphone types and the fact that both smartphones ran the identical motion measurement app, it is believed the impact on the data is negligible.

By performing analysis of motion data, it was shown that individual runners exhibit unique features that are expressed through the acceleration and rotation of their wrists and ankles while running. The runner comparison algorithm involving diffusion maps and ergodic measures achieved a high success match rate. In addition to the runner comparison algorithm, and in accordance with an early embodiment of the present invention, an iPhone motion measurement app was produced to collect motion data, as well as extensive MATLAB code implementing the algorithm and data analysis techniques described. The iPhone app captures limb activity data using the sensors built into the phone, places a time-stamp into the data stream, and continuously transfers the time-stamped data to the cloud. The data on the cloud are continuously analyzed using the methods described above and a quantification of limb activity data is produced in real-time.

This experiment demonstrates clear success in determining a specific limb activity signature for each individual based on diffusion map analysis of ankle and wrist activity data. In accordance with a disclosed embodiment of the present system 10 it is noted that data is aggregated from multiple days and weeks of monitoring, to build a detailed patient and limb specific activity signature. The limb activity signature consists of a continuously updated activity map of the patient's limb activity. The signature is qualified by patient state, time of day, time of year, and geographical location. Deviations from this activity map are continuously obtained using a running window over past activity. The running window is compared to the signature. Deviations of the measurements within this running window are qualified by the magnitude of the deviation and the duration of the deviation. The length of the running window is determined for each patient. The running window is expected to be 10 minutes in duration for most individuals. For more sedentary individuals, or time-periods when an individual is sedentary, a longer running window is used.

Ultimately, and in accordance with a disclosed embodiment of the present invention, the evaluations are performed in three distinct manners. First, the evaluations are performed in the ward on patients who have recently suffered a stroke. These patients are studied over a few days while they are in the hospital. The limb asymmetry in these patients is resolved, to a degree, over the duration of their stay in the hospital. This restoration of function is measured, towards baseline. The time-line is then reversed and these measurements are used as a surrogate to develop the asymmetry signature, and quantify the magnitude of the deficit which results from a stroke. Second, patients are tested at a neuro-rehabilitation center. These are patients who have had a stroke. Patients are at the greatest risk of a subsequent stroke in the first 90 days after a stroke. These patients are measured in a controlled environment 24 hours/day for up to 90 days. Third, individuals are tested in their natural environment. In the second and third distinct manners for evaluation the goal is to detect stroke occurrence in near real-time (within 10-30 minutes of the occurrence of a stroke).

### C. Patient Management System

Considering a disclosed embodiment of the present invention, in conjunction with data developed by the acquisition system 40 and the information generated by the analysis system 44, the patient management system 46 identifies anomalies indicative of patient specific alert conditions. Upon the identification of a patient specific alert condition, the patient management system 46 performs various functions.

In particular, and most importantly, the patient management system 46 directly interacts with the patient 100 to notify the patient 100 that a patient specific alert condition has been detected. In accordance with a preferred embodiment, and as mentioned above, the patient is contacted via the communication device 10d, which in accordance with a preferred embodiment may be the smartphone 35 that is also functioning as the data transmission device 16.

The first contact with the patient 100 is used to determine if there are any untoward situations which have arisen, which resulted in the alert condition. This first contact is used to rule out conditions such as non-working body worn sensors 12, or if the patient 100 has been unusually still for any reason other than normal behavior. The patient 100 is asked to perform a standard movement with each set of limbs. This standard movement task could be as simple as standing up and raising each arm. This interaction allows the operator of the system 10 to determine the responsiveness of the patient 100, and score their response to a standard test, akin to a neurological exam. Additional evaluations may include analysis of audio and of facial muscles to detect changes reflective of stroke.

Once it is determined that further action is necessary or if the system 10 fails to achieve interaction with the patient 100, the patient's caretaker(s) 102 is notified. The caretaker(s) is provided with detailed information on patient location, alert condition, the time of the attempt to interact with the patient, and the result of the attempt to interact with the patient. If the patient is outside a facility, then the patient management system 46 also, optionally, provides the caretaker(s) 102 with information on the nearest facility where the patient can be taken, and alert the staff at the facility as to the condition of the patient, and the alert condition which was generated.

### IV. DETECTION OF STROKE AND THE SEVERITY OF STROKE

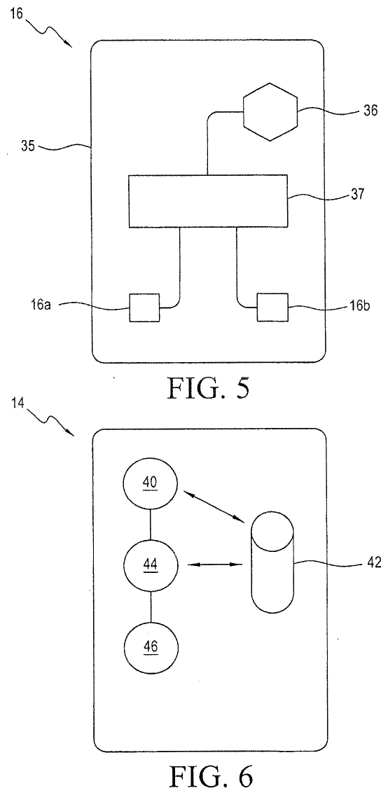

As discussed in disclosing the embodiment presented above, the expression of the motor control system is captured through placement of sensors on the body. Of particular interest are inertial measurement unit (IMU) sensors used to capture the movements of the upper limbs. In accordance with a disclosed embodiment, these include sensors placed on the legs, wrists or on the fingers. These sensors can be 3-axis accelerometers or 6-axis or 9-axis sensors with the additional sensors capturing 3D gyroscope and magnetometer readings. The movement data captured for an individual exists in a multidimensional coordinate space representing sensor readings captured from different points of an individual's body. The embodiment disclosed above with reference to FIGS. 1 to 11 demonstrates that there is an inherent structure to these data which capture the essence of the motor control system of the individual. This structure may not be readily visible in sensor space because each sensor captures just a bit, and perhaps an incomplete bit, of the expression of the motor control system. However, through an appropriate mapping the sensor data is transformed to a second space where the inherent structure is clearer. Further, it is appreciated that this structure represents an approximate model of the composite whole of the above-described motor control system. The expression of the motor control system, through the mapping, describes a geometrical surface, that is, a manifold. This geometrical structure is necessarily complex in normal individuals because it defines the full richness of human movement. The movements are mapped on to a geometrical structure, a manifold which captures amongst other aspects the control, expression, synchrony, balance, readiness to act, and range of symmetry and asymmetry of an individual's movements.

As described with reference to the embodiment of FIGS. 1 to 11, diffusion maps are used to uncover a manifold which captures the dynamics of an individual's movements. That is, the expression of the human motor control system can be characterized by the geometrical structure of the data obtained through the diffusion maps method described by Coifman and Lafon [Coifman, R. R. and S. Lafon, Diffusion maps. Applied and Computational Harmonic Analysis, 2006. 21 (1): p. 5-30]. Further, the manifold revealed is individual and can be considered as that person's movement signature. While the disclosed embodiment uses diffusion maps for manifold learning, other methods for manifold learning have also been found to be applicable.

The diffusion maps method allows the capture of an individual's movement signature and detection of the deficit introduced by a stroke or an injury. For purposes of illustration, consider, for example, the richness of movement of a ballerina or a sprint runner. Consider as well, an injury to the brain of such a trained individual. The injury will result in a loss to the richness of the movement for that individual, for example in the velocity, acceleration, range, fluidity, or symmetry of movement. This loss will be apparent to the naked eye because the individual would have otherwise been capable of executing fine practiced movements. An injury to the brain in other individuals also results in a loss of the richness of the movement of that individual. While this may or may not be apparent on visual examination, it can be measured as a loss of the richness of the geometrical descriptors of the data. That is, if normal expression is captured with a larger number of eigenvectors, the sensor signals under a deficit will be captured by a smaller number of eigenvectors. This change is detected and quantified as shown with reference to, for example, FIG. 11. FIG. 11 displays a loss in the richness of the geometrical descriptors of sensor data in the post-stroke condition. A stroke essentially reduces the dimensionality of the data captured by the diffusion maps method.

While the above disclosure focuses on the motor control system and the use of IMU sensors to capture the movement signature for an individual and the loss which results from an injury to the brain, it is appreciated this description can be readily extended to other brain functions and modalities of measurement. The above disclosure relating to FIGS. 1 to 11 can be extended, for example, to language and the use of audio sensors to capture the speech articulated, or language and the use of video to capture the movement of facial muscles. It can also be extended to modalities such as the galvanic skin response, heart rate variability, temperature, pressure, etc. It also extends to a combination of multiple such modalities within a single diffusion map evaluation. In general, brain injury results in a loss of function and a loss in the richness of the geometric descriptors of the data described by the diffusion maps method.

The present invention approaches this by building a monitoring system to analyze the user's movements and perform early detection of stroke by quantifying changes in the user's movements. As shown in the above description relating to FIGS. 1 to 11, the impact of injury results in a reduction in the richness of the movements of the individual. That is, compared to the movements before an injury there is a change in different aspects of sensor signals used to monitor an individual which reflect this reduction. In the above description relating to FIGS. 1 to 11 it was disclosed that these aspects could be discerned by tracking an ensemble of diffusion geometry measurements and time-series measures of signal magnitude, variability, complexity, and interrelation. The embodiment disclosed below with reference to FIGS. 12 to 37 builds on the above description relating to FIGS. 1 to 11 to show and apply the understanding that the greater the severity of the stroke the greater the resultant loss in the richness of the movement signals. This loss in the richness of the movement signals captured is discerned in accordance with the embodiment disclosed with reference to FIGS. 12 to 37 with enough specificity and sensitivity to create an approach to detect a stroke. Further, it is demonstrated that this can be performed with enough discrimination to determine the degree of the deficit which results from a stroke.

Referring to FIGS. 12 to 37, this embodiment is disclosed in detail. As will be appreciated based upon the following disclosure, this embodiment employs motion data that reflects upper limb movements of a user. The motion data are received from one or more sensors. Specific changes in user movement are determined by estimating several quantitative signal features of signal magnitude, variability, complexity, and interrelation. As a next step these quantitative signal features are input into machine learning models to detect if the user's movements reflect a change due to the occurrence of a stroke, that is to classify the data as being normal data or stroke related data. The quantitative signal features are also used with machine learning models to determine the degree of motor deficit induced by a stroke. The solution provided by this embodiment operates in two distinct modes, one by continuously monitoring subject activity using a moving window approach and the second by evaluating short duration data segments when the subject is performing prescribed movement tasks. In both modes the solution detects if the user has suffered a stroke and estimates a motor deficit score to determine the severity of the stroke.

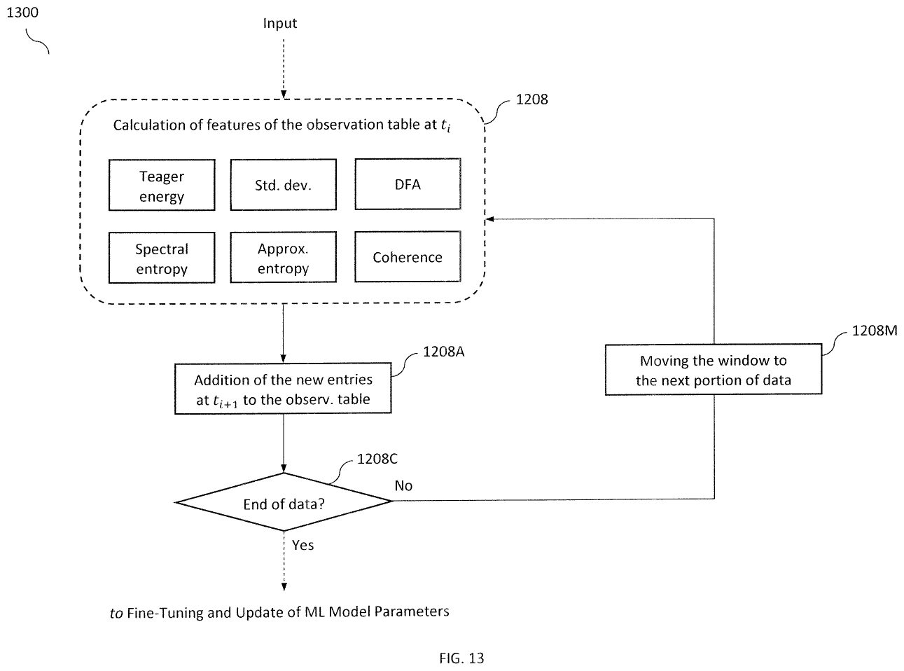

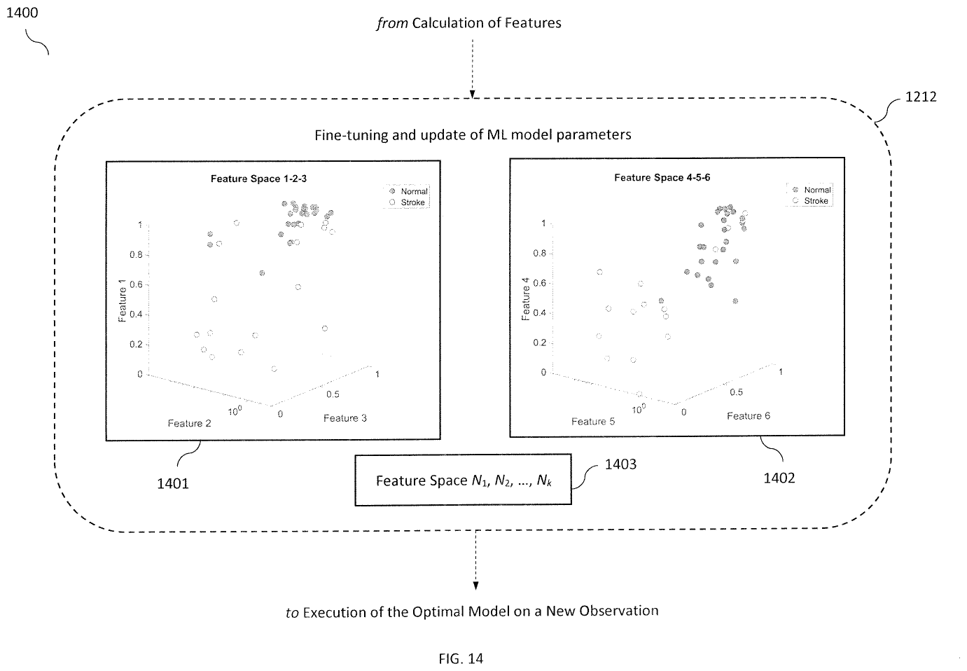

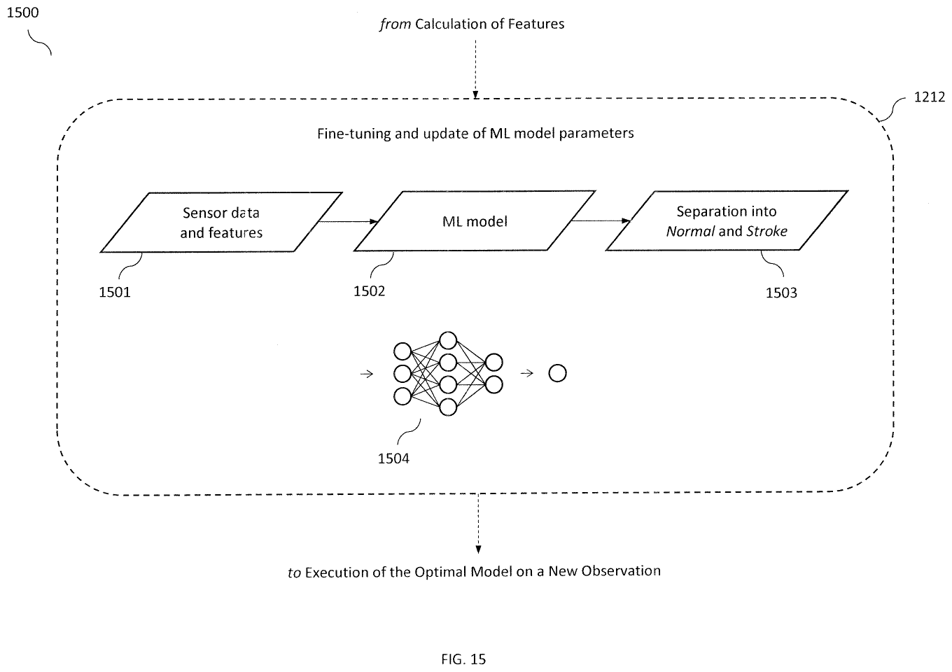

In accordance with the disclosed embodiment, the general architecture of a system 1200 for monitoring and alerting for strokes is shown in FIG. 12. The system 1200 includes (1) wearable devices 1201a, 1201b with motion sensors 1202a, 1202b, (2) data 1203a, 1203b recorded by the motion sensors 1202a, 1202b, (3) a computational method 1205 and (4) machine learning models 1211. The computational method 1205 and the machine learning models 1211 operate in two different modes, analyzing continuous data or analyzing task-specific data. The computational method 1205, as illustrated by schema 1300 in FIG. 13, includes the calculation of a number of features 1208 for a given segment of the data 1203a, 1203b, with the results 1208A being saved and subsequent data processing with a moving window 1208M until sufficient data has been processed 1208C to quantify activity and transmit the measurements to machine learning models 1211. Further, as illustrated by a general schema 1400 in FIG. 14, a fine-tuning of the machine learning models 1212 is performed to find the optimal combination(s) of the calculated features among all possibilities 1401, 1402, 1403. The aim of the fine tuning is conceptually illustrated by a general schema 1500 in FIG. 15, starting from the data and features 1501, through the multiple iterations of training and validation of a machine learning model 1502, and resulting in a classification decision 1503 possibly using a deep learning neural network 1504.

In some implementations the system 1200 is a client-server solution, where the wearable devices 1201a, 1201b with the sensors 1202a, 1202b are deployed on the user (or client) side, while the computational method 1205 and the machine learning models 1211 are executed on the server side using one or more computers 1204 (e.g., servers). The servers 1204 may be implemented locally on premise or on the cloud in one or more locations. In a client-server implementation of the system 1200 the data 1203a, 1203b is transmitted from the user to the server 1204 by a communication means 1218 (e.g., a smartphone) via a network 1217 (e.g., wi-fi, a cellular network, etc.). In the same manner, the notification of detections 1216 is transmitted back from the server 1204 to the user's device 1201a (or 1201b). Also, in some implementations, if the subject has been found to have possibly suffered a stroke, the notification 1216 may additionally be transmitted to receivers that comprise one or more separate entities, e.g., the individual's caretaker 1219, an emergency response system 1220, etc. A client-server architecture may be needed for various reasons, e.g., if the computational method 1205 and/or the machine learning models 1211 cannot be executed on the wearable devices 1201a, 1201b or the smartphone 1218 because of limited computational capacity or limited battery life, etc. However, with increase in computational ability and optimal selection of computational kernels some of the cloud or server-side functionality of the system 1200 can be deployed on the smartphone 1218 or on the wearable devices 1201a, 1201b. That is, the computation can be increasingly moved to the edge and out of the cloud with advance in computational capability.

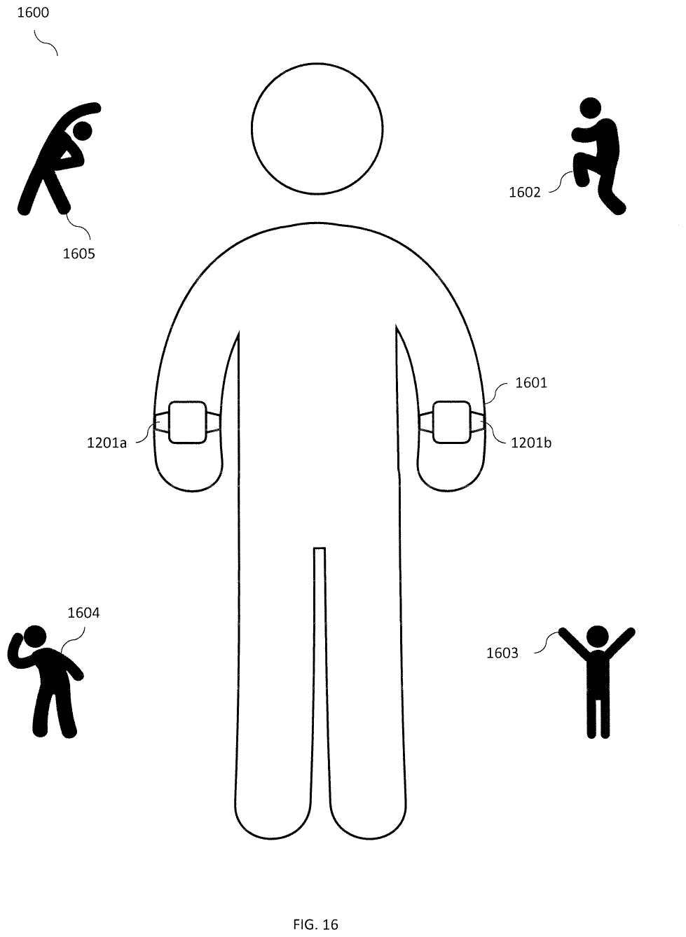

In the continuous monitoring mode, the data 1203a, 1203b is an extended dataset reflecting the user's activities of daily life, e.g., walking, lying, sleeping, sitting, playing etc. Examples of activities of daily living, such as standing 1601, running 1602, greeting 1603, talking on the phone 1604, exercising 1605 etc., are shown in FIG. 16 as an overview 1600. In the task-driven mode the data 1203a, 1203b are finite datasets recorded as a result of the user performing a number of prescribed upper limb movement tasks, e.g., lift your arms out to your sides and do five small arm circles, trace a circle in the air with both hands, etc.

As discussed above, in accordance with a disclosed embodiment and as discussed below with reference to FIGS. 12 to 37, the wearable devices 1201a, 1201b with an example of a sensor-related coordinate frame XYZ are shown in FIG. 17 as a general schema 1700. While the sensors 1202a, 1202b of the wearable devices 1201a, 1201b collect the data 1203a, 1203b, these are recorded with respect to the coordinate frames of the sensors 1202a, 1202b which are continuously changing their orientation in space. For each component of the acceleration vector the data 1203a, 1203b is being recorded. In the implementation described here the sampling rate is at least 12.5 Hz. Although in FIG. 17 the sensor-related coordinate frame is illustrated to contain three axes, which implies recording a three-dimensional acceleration vector, the sensors 1202a, 1202b can also measure orientation in space, thus recording from one to three angular rotations. In some implementations the sensors 1202a, 1202b can also measure magnetic field around the device, thus recording, depending on the technical characteristics, the direction, strength, or relative change of the field.

The firmware on the wearable devices 1201a, 1201b supports several features to maintain accurate continuous monitoring for extended periods of time extending to days, weeks, and months. Two of these features are key. First, the accuracy of a real-time clock on a wearable device depends on several factors including stability of the power supply, quality of the clock crystal, and the temperature of the crystal. The clock on current wearable devices can drift and be either advanced or delayed by several seconds in a day. Since we use multiple devices 1201a, 1201b, a smartphone 1218, and a cloud backend 1204, which all have independent clocks, this can result in a lack of synchrony. This is most acute for the clocks in the wearable devices 1201a, 1201b and the synchronization of the data 1203a, 1203b from them. A solution has been developed to keep the device clocks synchronized. In accordance with a disclosed embodiment, the smartphone clock is considered to be the master clock and constantly measures and corrects wearable device clock drift with respect to the smartphone clock. This solution maintains very close synchronization (within a few milliseconds) of the wearable device 1201a, 1201b and smartphone clocks indefinitely. Second, the wearable devices 1201a, 1201b communicate with the smartphone 1218 over a radio link (e.g., Bluetooth low energy (BLE)). Given the constraints of a wearable form-factor on the antenna and radio power, BLE is susceptible to dropped links. In the event of a dropped link most devices require user intervention to re-create the communication link between a wearable device and smartphone. This issue is addressed by constantly monitoring the communication link and seamlessly re-negotiates it if the link is lost. This re-negotiation does not require user intervention and is handled in the background. In the event of a dropped BLE link, the data 1203a, 1203b is buffered on the wearable devices 1201a, 1201b, with data being transmitted to the smartphone 1218 and cloud 1204 in the background once the link is re-established. That is, the device firmware ensures no data is lost if the radio link is dropped.

### A. Measures of Signal Magnitude, Variability, Complexity, and Interrelation

The computational method 1205 estimates the signal features 1208 by processing the data 1203a, 1203b. The raw data and the features are input into machine learning model 1211 for training and into the optimized machine learning model 1215 to classify the subject state. The classification identifies activities of daily living, distinguishes between normal and stroke states and determines the severity of a stroke. The system tracks an ensemble of diffusion geometry measurements and other time-series measures of signal magnitude, variability, complexity, and interrelation determined from the measurements of the body-worn sensors. The data are analyzed using time-series analysis measures: non-linear energy (NLE) as a measure of signal magnitude, approximate entropy (ApEn), standard deviation, detrended fluctuation analysis (DFA) and spectral entropy as measures of signal variability and complexity, and coherence and cross-ApEn as measures of interrelationship.

### 1. Nonlinear Energy

In addition to traditional measures of signal magnitude we use signal energy measured by non-linear (Teager) energy (NLE) [Kaiser, J. F., On a simple algorithm to calculate the “energy” of a signal, in Proceedings of ICASSP. 1990. p. 381-384. Zaveri, H. P., et al., A decrease in EEG energy accompanies anti-epileptic drug taper during intracranial monitoring. Epilepsy Res, 2009. 86 (2-3): p. 153-62.]. The conventional measure of signal energy weights contributions from all frequencies of a signal equally. Teager argues that while this is appropriate for electrical circuits, it is not appropriate for many physical systems, because greater energy is required in these systems to generate activity at higher frequencies. NLE, based on a model of simple harmonic motion, is a weighted measure of signal energy (E∝w²/A², where A is signal amplitude and w is frequency) such that high-frequency signals contribute more to the measure of energy than low frequency signals.

### 2. Statistical Measure of Variability

The standard deviation of the time series estimated as σ=std x is used as a statistical measure of variability.

### 3. Detrended Fluctuation Analysis

Detrended fluctuation analysis, related to the Hurst exponent, is a measure of the self-similarity, or long-term memory, of a time series. The detrended fluctuations analysis exponent is

$$H \sim \frac{log Δ x}{log Δ t} .$$

### 4. Spectral Entropy

Signal complexity is estimated in the spectral domain as spectral entropy and features of spectral entropy are determined such as the mean of spectral entropy

$$S {E}_{E} = \frac{1}{N} {\sum }_{t = 1}^{N} {\sum }_{f} {p}_{i} ( f ) log {p}_{i} ( f ) ,$$

and the standard deviation of spectral entropy SE_{σ}=std Σ_{f} pᵢ(f)log pᵢ(f).

### 5. Approximate Entropy and Cross Approximate Entropy

ApEn is a nonlinear measure that quantifies the complexity of a time series and is independent of signal amplitude. A high ApEn value indicates a complex or irregular time series, while a low ApEn value indicates a more regular and predictable time series [Pincus, S. M., Approximate entropy as a measure of system complexity. Proc Natl Acad Sci USA, 1991. 88 (6): p. 2297-301. 5. Joshi, R. B., et al., Regional and network relationship in the intracranial EEG second spectrum. Clin Neurophysiol, 2016. 127 (11): p. 3485-3491]. While ApEn measures the complexity of a single time series, cross-ApEn measures the degree of synchrony between two time series [Pincus, S. M. and B. H. Singer, Randomness and degrees of irregularity. Proc. Natl. Acad. Sci., 1996. 93 (5): p. 2083-2088].

### 6. Coherence

Coherence, the normalized cross power spectrum, is complex-valued and also independent of signal amplitude [Carter, G. C., C. H. Knapp, and A. H. Nuttall, Estimation of the magnitude-squared coherence via overlapped fast Fourier transform processing. IEEE Transactions on Acoustics, Speech, and Signal Processing, 1973. 21 (4): p. 337-344. Carter, G. C., Coherence and time delay estimation. Proceedings of the IEEE, 1987. 75 (2): p. 236-255. Zaveri, H. P., et al., Measuring the coherence of intracranial electroencephalograms. Clin Neurophysiol, 1999. 110 (10): p. 1717-25.]. Magnitude-squared coherence (MSC) can be interpreted as a frequency-indexed correlation coefficient. At a given frequency, MSC is a bounded measure (0<=MSC<=1), and MSC=1 if and only if there is a linear relationship between two random processes at that frequency. Features from coherence are determined such as the maximum

$$M S C {C}_{max} = {max}_{f} M S C ( f ) ,$$

and the frequency of the maximum MSC fₘₐₓ={f:MSC (f)=Cₘₐₓ}

### 7. Univariate and Bivariate Measures

Importantly, NLE, standard deviation, DFA, spectral entropy and ApEn are univariate measures. They are estimated for one time series at a time. Coherence and cross-ApEn are bivariate measures. They are evaluated for a pair of time series to provide measures of the interrelationship of the two. The pair could be formed from two time series obtained from a single wearable device, or from time series from two wearable devices. As an example, an instance is considered where two wrist-worn devices are employed, one on each arm. Each wrist-worn device measures one or more modalities. At the most elemental a 3-axis accelerometer is defined on each wrist, that is six time series are measured, three from the left arm and three from the right arm. For purposes of explanation, these time series are labeled LX, LY, and LZ for the three axis measurements from the left arm and RX, RY and RZ for the 3-axis from the right arm.

Univariate measures can be estimated for each of these six time series. One can, as an example, consider a summary measure for an arm formed as the average, at every point in time, for the three time series from the left arm, and for the three time series from the right arm. The summary measure for the left arm can be compared to that for the right arm to assess symmetry. This example is not meant to be limiting, as it is appreciated one could, for example, use measures other than the average to combine the three measures from an arm. Regarding the bivariate measure, the pairwise measurements are assessed as shown in Table 1. There are 15 pairwise evaluations in the upper part of the matrix. Further, there are three kinds of pairs which are formed: first, for the three pairs formed between the left 3-axis IMU sensor time-series (LX×LY, LX×LZ, and LY×LZ); second, for the three pairs formed between the right 3-axis IMU sensor time series (RX×RY, RX×RZ, and RY×RZ); and third, for the nine pairs formed between the left and right sensors (LX×RX, LX×RY, LX×RZ, LY×RX, LY×RY, LY×RZ, LZ×RX, LZ×RY, LZ×RZ).

As another example, the three pairs formed between the time series on the left arm can be averaged to form a summary of interaction terms for the left arm (denoted here as L×L). The three pairs formed between the time series on the right arm can be averaged to form a summary of interaction terms for the right arm (R×R). The nine pairs formed between the time series on the left and right arms can be averaged to form a summary of interaction terms for the two arms (L×R). For measures such as MSC where directionality is not important, we will assess only the pairs shown in either the upper or lower triangular part: because they are the same, it does not matter which one is used. For bivariate measures such as cross-ApEn where directionality is important, both the forward and reverse directions are evaluated. That is, the measures for both the upper and lower triangular parts of the matrix shown in Table 1 are evaluated.

For given data 1203a, 1203b, the computational method 1205 comprises the calculation of nine types of features: NLE, standard deviation, DFA, ApEn, mean of spectral entropy, standard deviation of spectral entropy, maximum coherence, frequency of the maximum coherence, and cross ApEn. The computational method 1205 may calculate these features after pre-processing the inputs, e.g., calculating the standard deviation of increments of the signal, mean deletion, de-trending, filtering etc. The data 1203a, 1203b can also be preliminarily transformed into a coordinate frame different from the one in which it is originally recorded before it is processed. Besides, it can apply additional filtering afterwards, e.g., smooth several successive values of a feature within a given segment when processing the signal in a moving window. In some implementations the computational method 1205 can derive other features from these measures, as well as use additional features. Diverse combinations of features, e.g., the ones based on specific components of the data 1203a, 1203b, can be used as well.

Table 1. Time series will be collected from multiple body-worn sensors. At a minimum we will acquire time series from two 3-axis IMU sensors, one each placed on the left and right wrist. The time series from the left 3-axis IMU sensor are denoted LX, LY and LZ. Similarly, the time-series from the right 3-axis IMU sensor are denoted RX, RY and RZ. We form bivariate measures between each pair of time series.

| | Data | | | | | |
|---|---|---|---|---|---|---|
| Evaluation | LX | LY | LZ | RX | RY | RZ |
| LX | | LX × LY | LX × LZ | LX × RX | LX × RY | LX × RZ |
| LY | LY × LX | | LY × LZ | LY × RX | LY × RY | LY × RZ |
| LZ | LZ × LX | LZ × LY | | LZ × RX | LZ × RY | LZ × RZ |
| RX | RX × LX | RX × LY | RX × LZ | | RX × RY | RX × RZ |
| RY | RY × LX | RY × LY | RY × LZ | RY × RX | | RY × RZ |
| RZ | RZ × LX | RZ × LY | RZ × LZ | RZ × RX | RZ × RY | |

### B. Machine Learning Models

Multiple machine learning models are created for detecting activities of daily living, detecting stroke, determining the severity of stroke from continuous data, and determining the severity of stroke from task-based data.

The computational method 1205 yields a table of observations which is used to train a machine learning model. For n calculated features and m moving-window-produced segments the computational method 1205 forms an m×n matrix—a user matrix—quantifying the behavior of the user in question.

The machine learning model for the detection of stroke is trained by classifying the data into either two or several classes. When two classes are specified the classification results in a binary output of normal or stroke. In the case where several classes are specified the classification results in a multinomial output, with class labels normal, mild, moderate, and severe representing different degrees of stroke related deficit. The assignment to one of the classes is based on empirical evidence that within certain ranges of values the above-given features of the multi-sensor data are specific for different degrees of motor deficit. The observation table used for the machine learning model provides a binary or multinomial determination and compares the output with the true classification label, so that the classification accuracy of the machine learning model can be determined by the resulting confusion matrix.

The procedure described above is repeated for continuous monitoring and task-based data to create separate models for continuous monitoring and task-based approaches.

In a separate evaluation a model is trained to determine activities of daily living from continuous data. Here, the measures are ascribed to one or more classes to determine which activity of daily living the user is engaged in. The model is created in a manner similar to that described above for multinomial classification for stroke severity, though now, the class labels are for different activities of daily living. Activities of daily living are used to define some of the meta parameters for the classifiers. The length of the moving window used for the classifiers is one such a meta-parameter. For example, if a person is asleep or sedentary a longer window is used and if the person is awake and active a shorter window is used to determine if a user has experienced a stroke or to evaluate the severity of the stroke.

The algorithm which raises the alarm can be composed of the following sub-parts. First is a comparison to a baseline signature for each arm, that is a comparison of each arm to the individuals' historical values for that arm. Second, is a comparison of one side of the body to the other side, that is a determination of asymmetry. Third, are versions of these models performed with an adjusted comparison, so that if a decision cannot be reached within a certain duration with a given approach and threshold, then additional data are used to see if a decision can be reached in a longer duration. The comparisons test whether the new signals recorded are within a predicted range or lie outside the predicted range. The method used to test also includes the use of previous and current data to predict new data, and the measurement of the error of this prediction. For example, historical and current data from the left limb could be used in conjunction with historical data from the right limb to predict new data for the right limb. If the error in this prediction is outside an expected set of values, then a detection is flagged. Similarly, historical and current data from the right limb could be used in conjunction with historical data from the left limb to predict new data for the left limb. If the error in this prediction is outside an expected set of values, then a detection is flagged. The threshold for reaching a decision is largest for shortest duration data and becomes increasingly smaller for longer duration data. If a decision cannot be reached within a certain longer time-period then the algorithm would move on to the next segment of data. The different methods would be trained with the individuals' data and will learn the individual's movement signatures and normal patterns with time, as illustrated in the general schema 1300 in FIG. 13, general schema 1400 in FIG. 14, and overview 1500 in FIG. 15.

A baseline signature starts to be formed as soon as the devices are placed on the individual and monitoring is initiated. The baseline signature is continually updated through monitoring. The baseline signature is defined as a function of subject state. That is, there is a different baseline signature for sleep and for wake, and for each activity of daily living. At the start of the monitoring the models would not be able to rely on historical data. At this time, the models would be informed by extant data from our database matching known aspects of the individual's age, gender, handedness, prior injury, or handicap, if any. Quick base line determinations can also be performed by assessing the individual in known movements, including task-based determinations which are describe below.

The determinations from the different features can also be evaluated separately and combined in multiple manners. First, if one or a small subset of the features is signaling a detection, then the algorithm goes into a mode where greater scrutiny of the data is warranted. On the other hand, if there is a detection by most of the features, then an alarm condition is met. Other methods also exist for combining detections with several features. For example, logistic regression, with step up or step-down methods to combine different features, can be used. There are multiple other options given the ongoing advances in modern data science methods for classification. These span the gamut from simple linear methods such as regression-based classification, logistic regression, decision tree and support vector machines (SVM) based methods. More current methods can also be used. These include deep learning methods with classical architectures such as convolutional neural networks (CNNs), recurrent neural networks (RNNs), the long short-term memory (LSTM) architecture or special network architectures such as deep and wide, skip or U-net architectures which allow a combination of deep layers with raw IMU data and feature values to classify data.

### C. Moving the Analysis Outside the Cloud

As indicated above, there are three parts to the solution provided by the present invention: body-worn sensors 1202a, 1202b, the data transmission device 1218 and the cloud-based analysis system 1204. The sensor data are acquired by the body-worn sensors 1202a, 1202b. They are placed into a packet with a timestamp from the real-time clock (RTC) on the wearable device 1201a, 1201b and transmitted from each body-worn sensor 1202a, 1202b to the transmission device or smartphone 1218. There a second set of packets is assembled, this time possibly with data from more than one sensor, with additional timestamps and information which aid in synchronizing the devices, smartphone and cloud solution clocks and maintaining the monitoring being performed by the overall system. This second set of packets is transmitted to the cloud solution. In the cloud the data are analyzed. This approach requires the transmission of sensor data to the cloud for analysis. As the computational capability, energy efficiency and battery life of wearable devices and smartphones improves, the computation of measures and algorithms upstream can be moved to these devices. This is done for the following reasons. First, as the computation is moved closer to the source of the data, the amount of data which must be transmitted through the data and computational pipeline is reduced and in doing so the power consumption is reduced, heat generation is decreased and battery life is increased. Second, as the computation is moved upstream, the latency with which decisions can be made is decreased. The further upstream the computation is performed, the lower the amount of data which must be transmitted from the wearable device to the smartphone and from the smartphone to the cloud and the faster a decision can be reached.

The most efficient computation is achieved when it is moved the most upstream. The NLE estimator, for example, is the simplest and most computationally efficient estimator employed. It requires a minimal number of subtractions and multiplications. The nature of this estimation is such that it can be performed in integer or float point math allowing a further reduction in the computational requirements as integer math is much more efficient than float point math. This computation can be moved upstream to the body-worn sensor. Now, there is a general sense that the more computationally efficient algorithms will be less powerful and perform with a lower accuracy. So, for example, the NLE evaluation is more computationally efficient but may have less accuracy than, for example, the ApEn evaluation. This means that the evaluation performed closest to the sensor may be less accurate, with evaluations being performed downstream being more computationally demanding but also more accurate. To manage this situation, it is important to note that there will be a need to periodically transmit full data through the computational chain so that all measures can be evaluated.

NLE can be estimated on the body worn device. The device for each arm would estimate the NLE value for its data, and this would be sent to the phone once every 30-seconds, for example. For each evaluation received the smartphone App could evaluate if an event could be ruled out or raw data were needed for more a complete evaluation of signal features. If more data were required, raw data would be transmitted from the body worn sensor to the smartphone.

One solution thus would be to have the NLE measure being estimated on the body-worn sensor; ApEn, standard deviation, DFA and spectral entropy measures being estimated on the smartphone; and coherence, cross-ApEn and diffusion maps evaluations being estimated on the cloud. With greater computational capability on the body worn and smartphone devices this could change, and more computationally demanding measures could be moved further upstream to the smartphone or the body worn sensor.

### D. Detection of Stroke and Quantifying the Severity of Motor Deficit During Continuous Monitoring

Example long-term recordings were acquired from patients who had suffered a stroke and were admitted to the Yale New Haven Hospital stroke center (YNHHSC). Data were also collected from age and sex matched normal subjects recruited from the Maplewood Senior Living (MSL) Center in Westport, Connecticut. The data from normal and stroke subjects consisted of continuous 3-axis IMU measurements acquired from the two arms. Data were acquired from more than 100 stroke subjects and 30 normal subjects. The subjects were provided with minimal instructions during data acquisition. Stroke subjects were monitored between 1-4 days and normal control subjects were monitoring for approximately 7 days. Ongoing activity recorded from stroke subjects and healthy control subjects was analyzed.

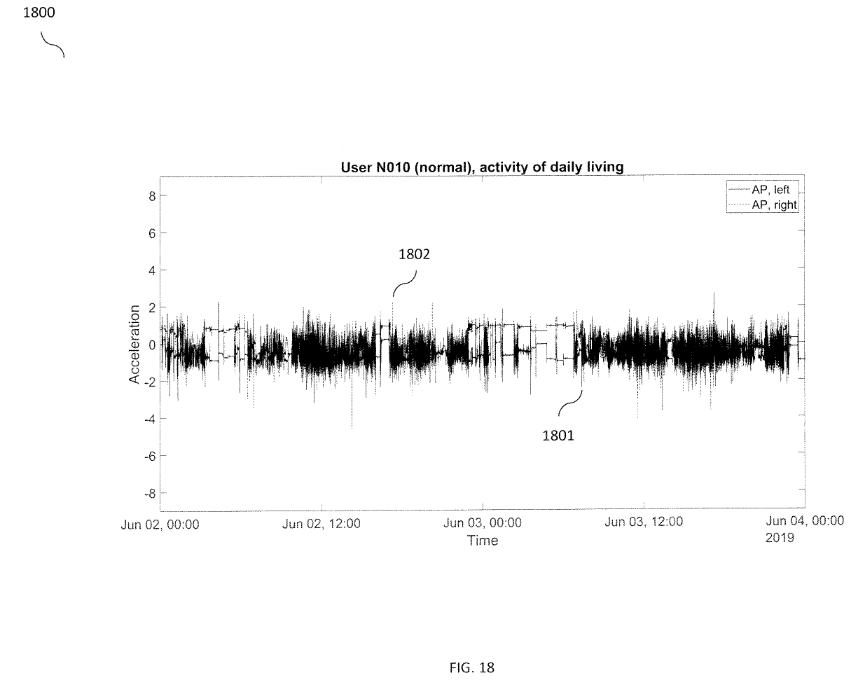

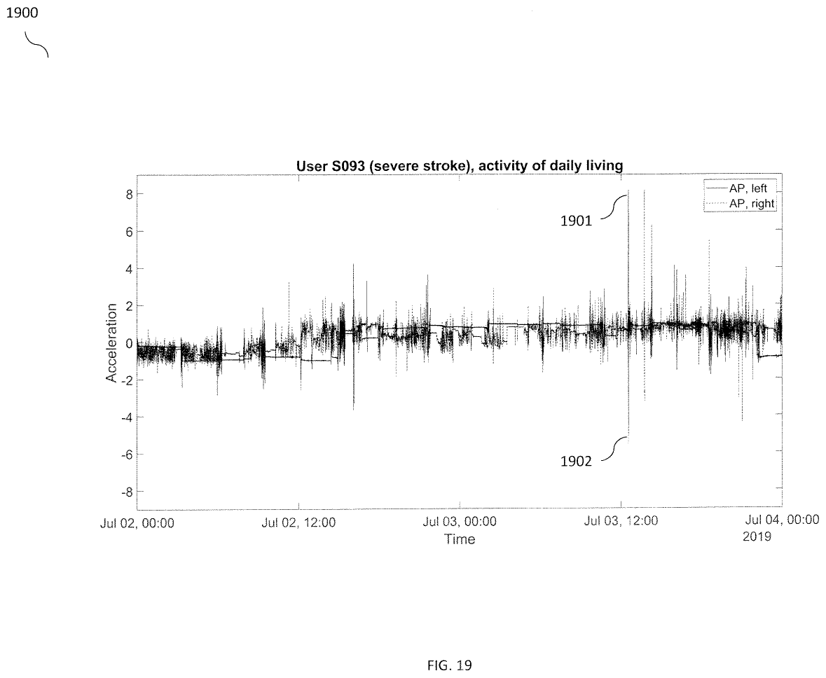

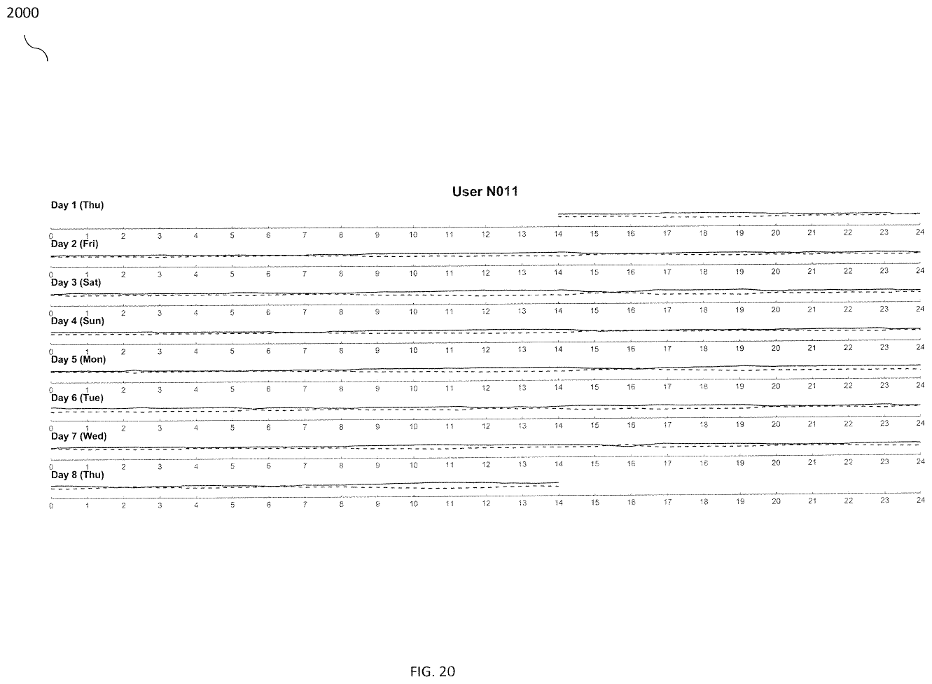

During continuous monitoring movement data is acquired as the user goes about activities of daily living, e.g., sitting, standing up, washing oneself, walking, running, cooking, eating, sleeping etc. FIG. 18, a 2D plot 1800, shows example data 1203a,b reflecting upper limb motion of a healthy control subject. Each of the two graphs displays temporal changes 1801, 1802 of a component of the acceleration vector recorded by two devices, on the left and right arms, while the subject is going about activities of daily living. As seen in FIG. 18, both graphs display complex behavior with considerable amplitudes. FIG. 19, a 2D plot 1900, shows example data 1203a,b reflecting upper limb activity of a stroke subject. Unlike the healthy control example, here the activity displays lower amplitude with sporadic outliers in the time series 1901, 1902.

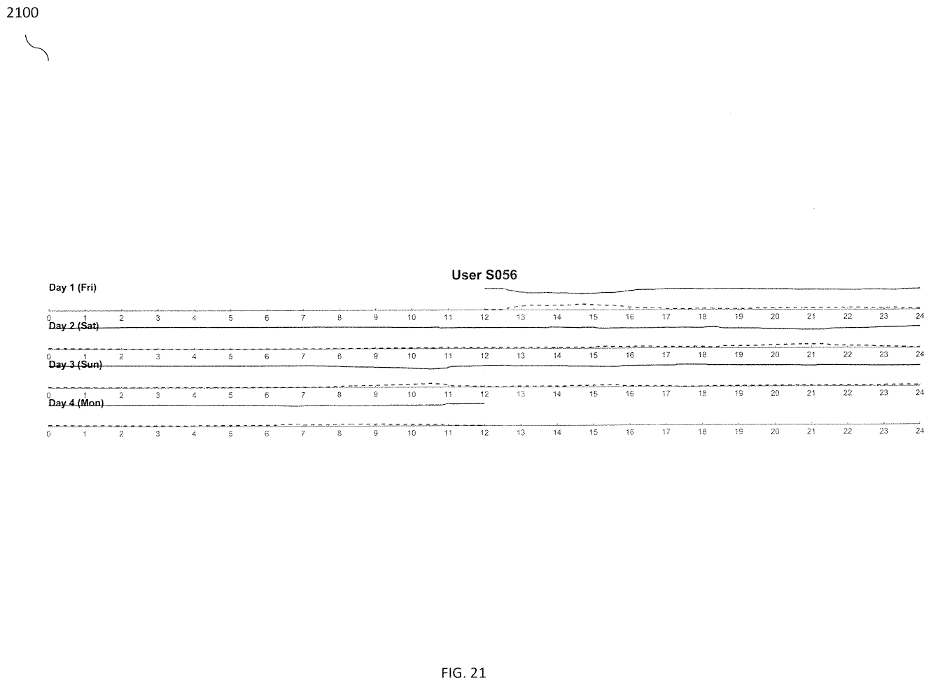

In FIGS. 20, 21 signal complexity is displayed for continuous data observed over multiple days in two subjects. FIG. 21, a 2D plot 2100, shows the evolution of ApEn, a nonlinear signal complexity measure, over four days (Friday to Monday) for a stroke subject. The ApEn estimates are plotted continuously, each subplot corresponds to a day, with the hour of the day shown on the X-axis. The Y-axis represents the dimensionless arm complexity measure as a ratio with respect to the other arm; the dashed trace shows Left/(Left+Right), while the solid trace shows Right/(Left+Right). The dashed trace is for the left (affected arm) and the solid trace is for the right (unaffected) arm. A clear and steady difference between the two curves was noted over the study period. These results make evident that the difference between affected and unaffected arms is striking and persistent through wake and sleep, resulting in a perfect separability between the signals from the two arms. In FIG. 20, a 2D plot 2000, signal complexity is displayed for an example healthy subject. This figure is like FIG. 21, but for data over 8 days (Thursday to Thursday). Unlike the stroke subject, here there is almost no difference between the measures of the left (dashed) and right (solid) arms, which results in zero false positive detections of stroke in these data.

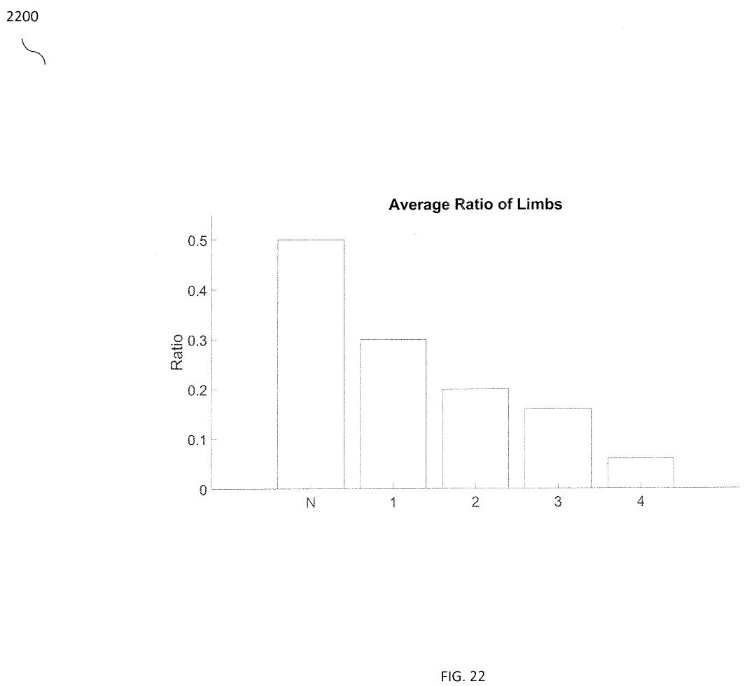

In FIG. 22, a bar chart 2200, the ability to capture the deficit resulting from a stroke is illustrated by using a measure of signal complexity (ApEn). The severity of stroke can be defined by the motor arm component of the NIH stroke scale (NIHSS). This scale ranges from 1 to 4 with 4 representing the most severe strokes. The results obtained in stroke subjects are compared to results from age matched normal volunteers. ApEn was estimated for each hour of the data and the results obtained in stroke subjects were compared to results from the normal volunteers. For normal subjects (labelled as “N”) the fractional value of ApEn was displayed for the left arm, that is, the ApEn for the left and right arms was estimated, and then the ratio Left/(Left+Right) was calculated. For stroke subjects, with NIH stroke scale NIHSS=1 to NIHSS=4, the fractional value of ApEn was plotted for the affected arm, that is, the ApEn was estimated for the affected and unaffected arms, and then evaluated the ratio Affected/(Affected+Unaffected). In this plot, a value of 0.5 would indicate perfect symmetry between the two arms. It is noted that there is a deficit which is increasingly more profound with increasing severity of stroke. The most profound loss of signal complexity is observed in individuals with a NIHSS of 4 (the most severe strokes). This result demonstrates an increasing loss of signal complexity with greater stroke severity.

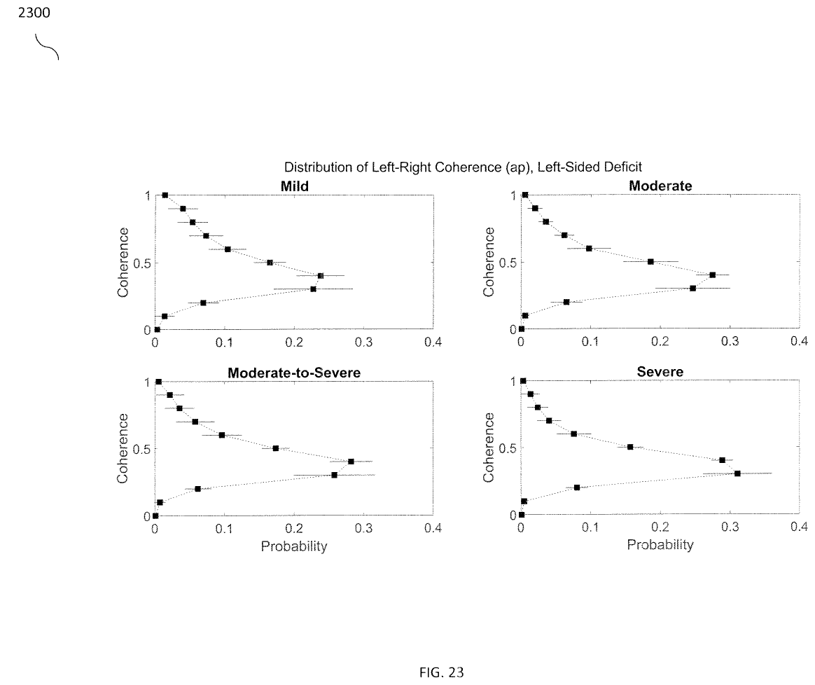

FIGS. 23-36 display selected computed features for a cohort of subjects who were categorized as having mild, moderate, moderate-to-severe, and severe stroke related deficits and were monitored continuously between 10 a.m. and 8 p.m. for between one to four days. In FIG. 23, in subplots 2300, the coherence between the left and right time series for the anterior-posterior (AP) component for all subjects is displayed. It can be observed that the more severe the motor deficit, the more likely the cross-limb coherence has values close to 0.

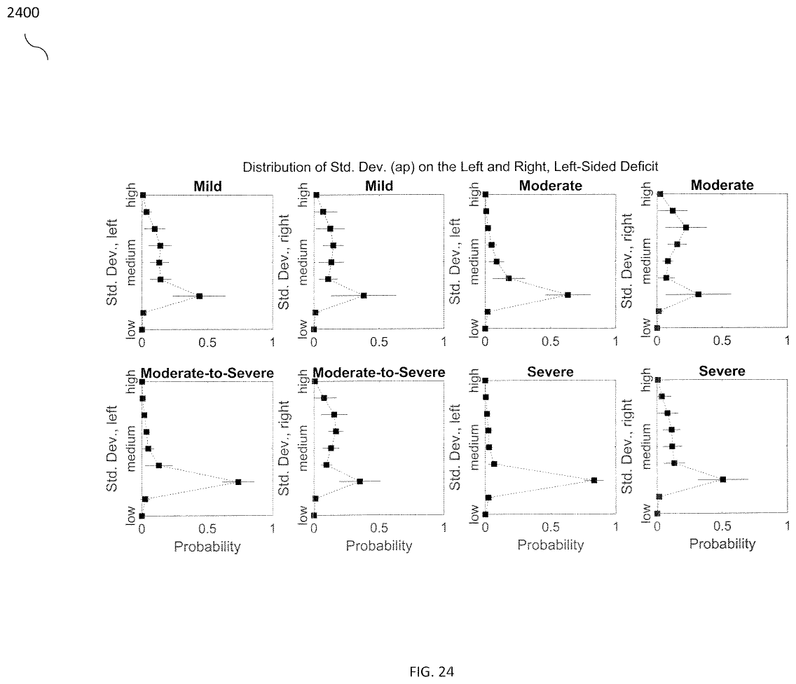

In FIG. 24, subplots 2400, the standard deviation of the AP component of the left and right limb time series for a cohort of subjects who experienced a left-sided deficit due to stroke is displayed. With increasingly greater stroke-related deficit it is observed that the standard deviation on the affected side of the body has increasingly smaller values, while on the healthy side the estimates of standard deviation remain close to normal.

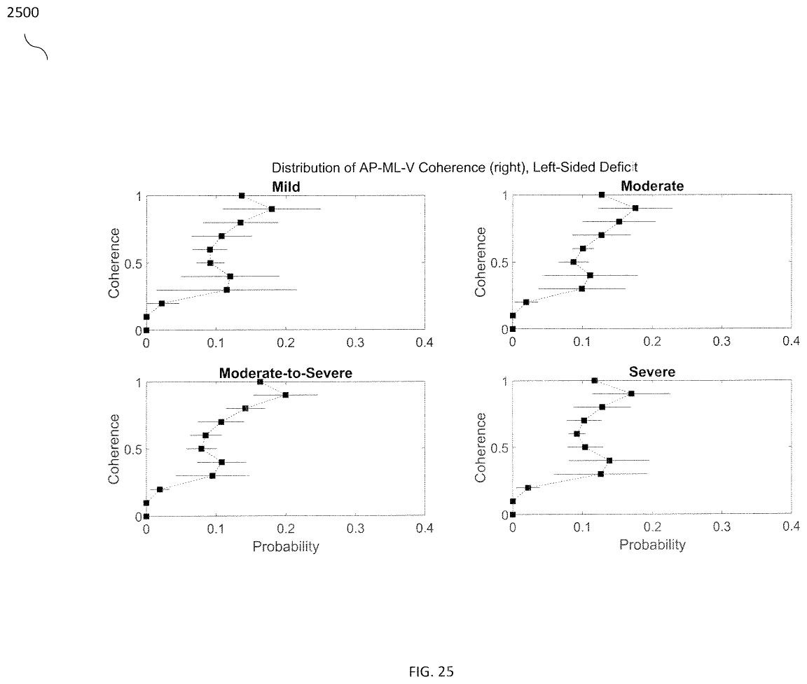

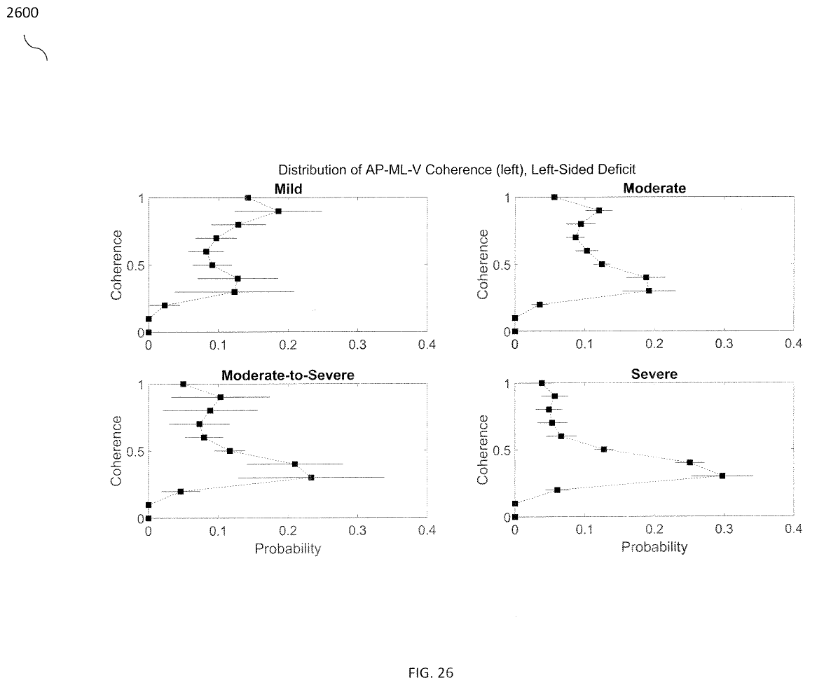

In FIGS. 25, 26, subplots 2500 and 2600, respectively, the coherence between the three acceleration components, AP, ML and V, for a given side of the body (within-limb coherence) is displayed. The subjects had experienced a deficit on the left side of the body. On the healthy side (right side of the body, FIG. 25) it is seen that the coherence demonstrates values which are close to the distribution of values observed for normal subjects and independent of the degree of stroke severity. On the affected side (left side of the body, FIG. 26), in contrast, it is observed that the within-limb coherence demonstrates smaller estimates with increasingly greater stroke-related deficit.

The analysis displayed in FIGS. 20, 21 and 23-26 indicates the ability of these computed measurements to distinguish between normal and stroke conditions, and between different degrees of stroke severity. The results demonstrate: (1) there is an underlying structure to the captured data, and that (2) increasingly more severe strokes result in increasingly greater signal change corresponding to an increasingly greater loss of signal complexity and change in inter-relationship. Individuals who experience a stroke are at a greater risk of experiencing one or more subsequent strokes, particularly in the first 90 days after a stroke. Because a graded difference can be detected using the approach of the present invention, it is appreciated that it will be able to detect a second or a third stroke as an increasingly greater deficit and thus detect not only the first, index, stroke experienced by an individual but recurrent strokes as well.

### E. Stroke Detection Using a Single Measure

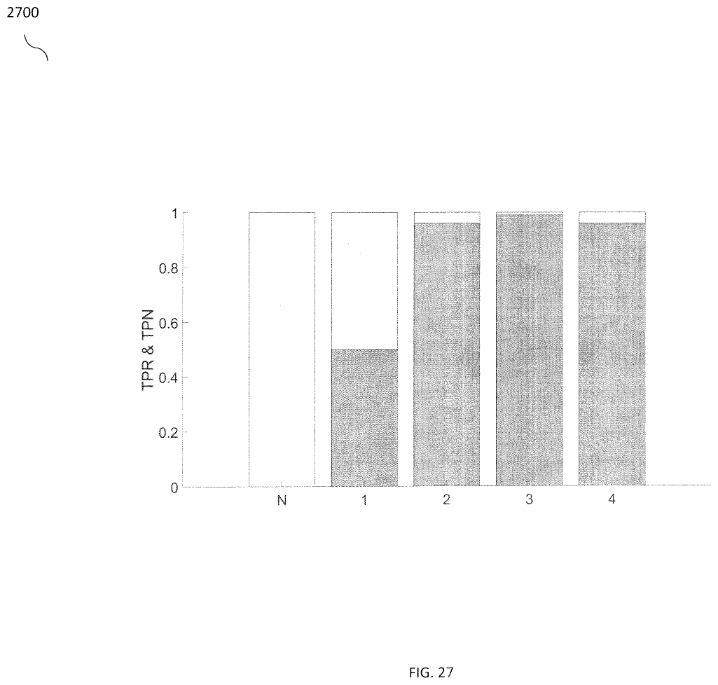

In FIG. 27, a bar chart 2700, the performance of the algorithm for detection of stroke when evaluated with a single measure, ApEn (signal complexity), is displayed. Sensitivity (or true positive rate, TPR) and specificity (or true negative rate, TNR) for detection of stroke were determined for a test data set. Sensitivity, or TPR, was evaluated from stroke subject data. Specificity, or TNR, was determined from normal subject data. The severity of stroke was defined by the motor arm component of the NIH stroke scale (NIHSS). This scale ranges from 1 to 4 with 4 representing the most severe strokes. Here, the dark gray shading indicates that a stroke alarm was raised and the light gray shading that an alarm was not raised. The data from normal subjects was correctly identified as being normal data most of the time and an alarm was not raised for these data. The TNR, estimated from the normal subject data, was 0.998, or there was one false positive every 21 days. For NIHSS (motor component) 1-4, the TPR was 0.50, 0.96, 0.99, and 0.96, respectively. A stroke with NIHSS=1 (motor component) is a mild stroke which does not necessitate treatment with tPA. For NIHSS 2-4 (i.e., for moderate to severe strokes which need timely intervention), the TPR was 0.97.

### F. Stroke Detection Using Multiple Measures

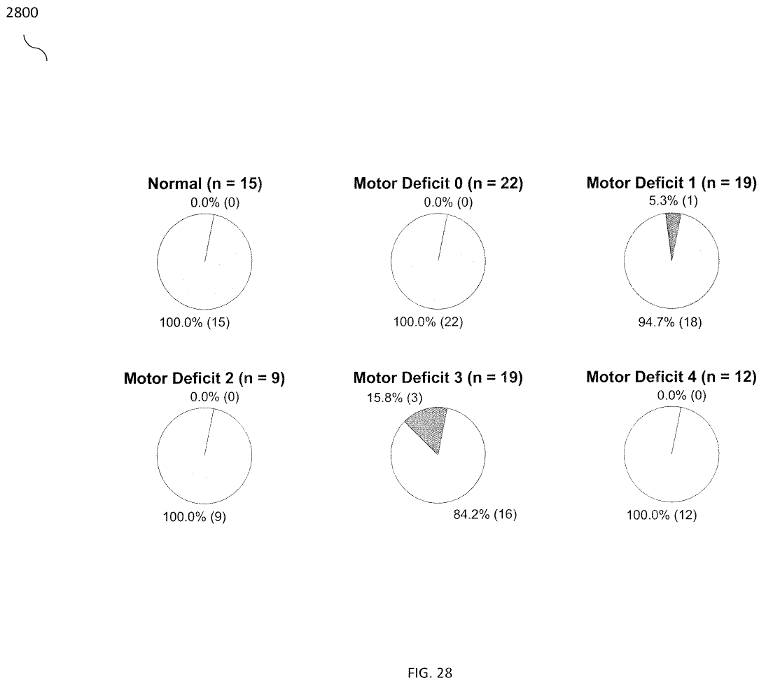

FIG. 28, pie chart 2800, shows an example of multinomial classification of data from normal subjects and from stroke subjects. The stroke data were labelled with five degrees of stroke severity, from quasi-normal to severe. The classification was obtained through a joint use of the features of within-limb coherence, DFA, ApEn and NLE. As seen in FIG. 28, the algorithm is perfectly accurate, because there are no false positives for non-stroke (i.e., normal) subjects and subjects with motor deficit difference 0 (quasi-normal), 2 (moderate) and 4 (severe). Overall, there were false positive classification decisions in only 4.1% of the subjects studied.

### G. Motor Deficit Assessment Using Multiple Measures

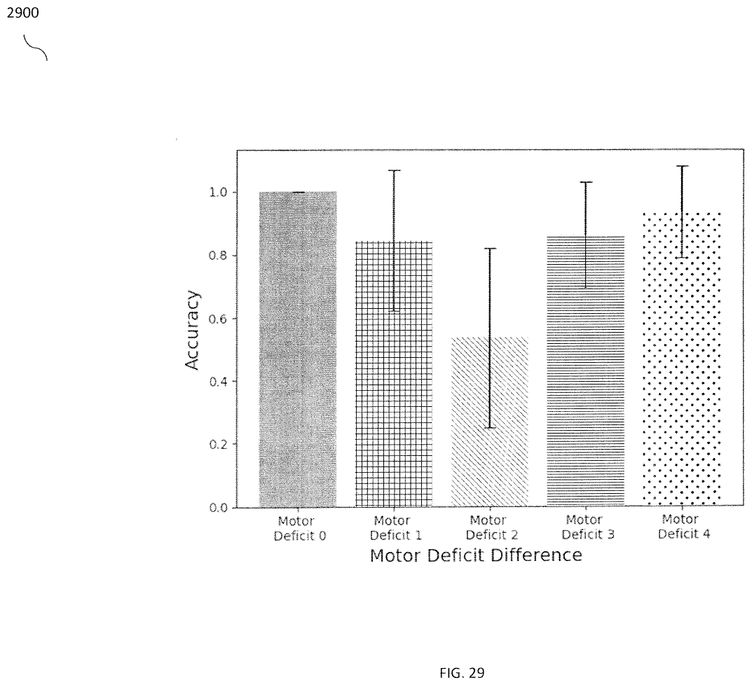

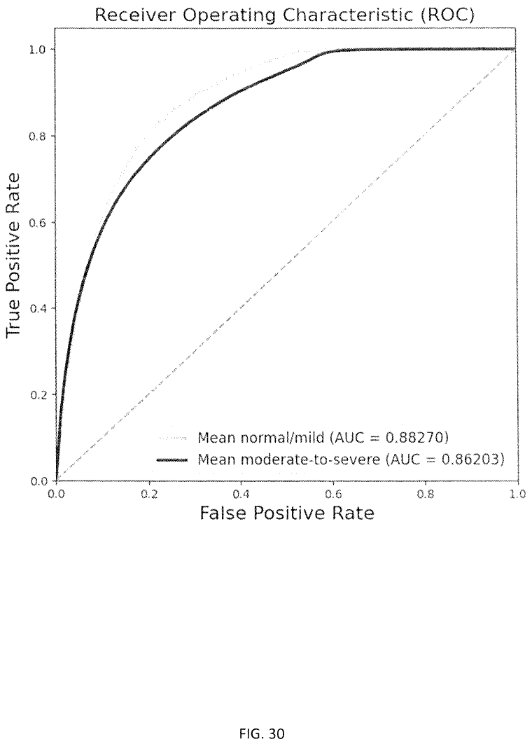

Given raw sensor data, a 1D CNN can be applied to process these sequential data to detect motor asymmetry or motor deficit difference between affected and unaffected limbs. This method can automatically extract features from raw data, capturing highly non-linear and complex patterns. Multi-binary classification framework can be adapted to enhance the performance and interpretability of traditional multinomial classification problems. Given five possible motor deficit differences between affected and unaffected limbs (normal: 0, mild: 1, moderate: 2, moderate-to-severe: 3, severe: 4) based on the motor arm component of the NIHSS, the problem was decomposed into a series of binary classifiers, each tasked with distinguishing between subsets of the corresponding classes, i.e. distinguishing 0 vs 1234, 01 vs 234, 012 vs 34, 0123 vs 4. Once the models are trained, the predictions from each model are aggregated to derive a final prediction. In FIG. 29, bar chart 2900, the models' aggregated accuracy for detection of motor deficit 0, 1, etc. is displayed using the multi-binary classification framework, i.e., detecting a particular motor deficit in comparison to all other possible motor deficits. For example, the value shown for motor deficit 1 represents the accuracy of detecting a difference of motor deficit score of 1 with respect to all other labels. FIG. 30, on a 2D plot 3000, displays the averaged receiver operating characteristic (ROC) for correctly detecting normal or mild deficit versus the rest, and the averaged ROC curve for detecting moderate to severe motor deficit versus the rest.

While our current approach leverages ensemble method (one-vs-rest) from multiple binary classification models to predict among multiple classes, there are alternative implementations where a single model is directly trained using multiclass labels. In these cases, the model learns to distinguish among all classes simultaneously using techniques such as softmax activation in the final layer and categorical cross-entropy loss. This multiclass approach can simplify the training pipeline and reduce inference time, though it may be less flexible than an ensemble method when dealing with class imbalance or when the learning task becomes more complex.

In some implementations, 2D CNNs can be applied to a feature matrix where each row represents moving window over time (e.g., a short segment of a signal), and each column represents a specific feature extracted within that window (such as statistical measures, spectral, complexity, and temporal information). This matrix can be interpreted similarly to an image, where the vertical axis is temporal (windows) and the horizontal axis is feature-wise. Applying 2D CNNs to this representation allows the model to learn localized patterns both across temporal and feature space, potentially capturing interactions or dependencies that might be overlooked by simpler models.

In some implementations ensemble methods can be constructed by combining different types of neural networks (e.g., recurrent neural networks, transformers) with traditional machine learning algorithms (e.g., random forest, gradient boosting machines, support vector machines) that operate on handcrafted features. This hybrid ensemble approach leverages the strength of both representation learning and domain-specific feature engineering. Neural networks can automatically learn hidden representations from raw or minimally processed data, while traditional algorithms can perform well on curated feature sets derived from domain knowledge. The predictions can be aggregated through techniques such as majority voting, weighted averaging, or stacking which can improve robustness and generalization compared to any single model.

H. Detection of Stroke and Activities of Daily Living, and Quantification of Severity of Motor Deficit from Task Based Data

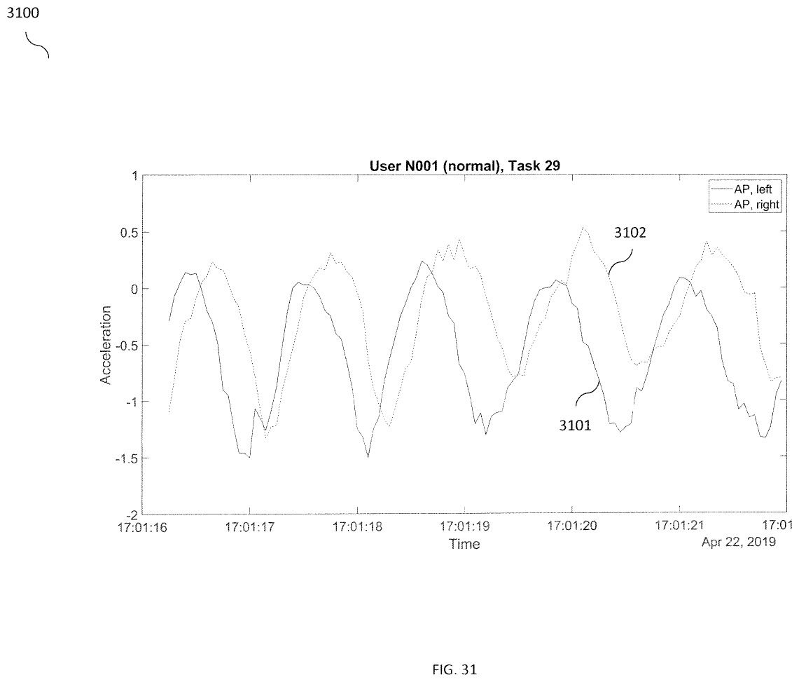

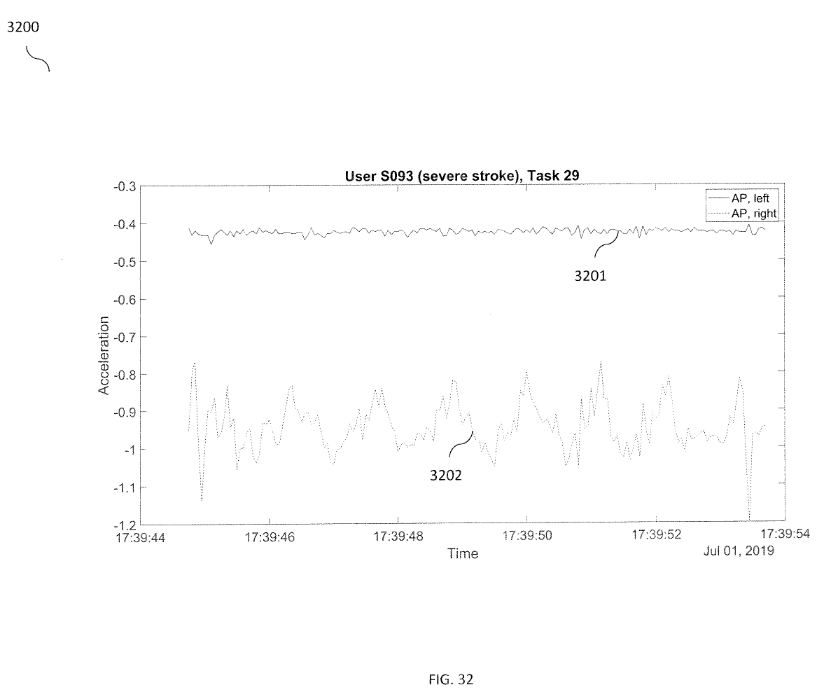

In some implementations the user is asked to perform a set of movements as prescribed by a specially developed battery of tasks (Table 2) to quantify the presence of motor deficit. The advantage of a task-based approach is that it provides a fast, reliable, and cost-effective screening of a subject. It requires minimal training and can be carried out by individuals who may not have expertise in diagnosing stroke. FIG. 31, 2D plot 3100, displays example data 1203a,b reflecting upper limb task-driven motion for a normal (healthy control) subject. Each of the two graphs represents temporal variations 3101, 3102 of a component of the acceleration vector recorded by two devices, one each on the left and right arms, respectively, while the user is performing a prescribed task. Both graphs in FIG. 31 display complex behavior with large amplitudes. FIG. 32, 2D plot 3200, in contrast, shows example data 1203a,b reflecting upper limb task-driven motion for a stroke subject. Here, unlike the healthy control subject, there is a considerable difference in the dynamics of the two graphs 3101, 3102. While one limb, 3202, demonstrates behavior similar to the healthy control case, the other limb, 3201, displays much smaller amplitudes and simpler dynamical changes.

Table 2. A battery of 38 tasks were designed by us to follow elements of the neurological exam. The tasks were designed to be easy to explain and perform.

| No. | Task | No. 2 | Task3 |
|---|---|---|---|
| 1 | Hold your arms out straight and don't let me bend your fingers | 20 | Remove your jacket and hang it on the back of the chair |
| 2 | Keep your fingers together and don't let me pull them apart | 21 | Wash your hands in the sink |
| 3 | Spread your fingers apart and don't let me push them together | 22 | Dry your hands using the towel |
| 4 | Turn your hands over and touch your thumb to the base of your pinky | 23 | Sit down |
| 5 | Lift your thumb up and don't let me push it down | 24 | Use a knife and fork to simulate cutting food |
| 6 | Make a fist and don't let me push your wrists down | 25 | Pick up the napkin with both hands and wipe your mouth |
| 7 | Push your wrists down | 26 | Pick up both paperclips at the same time |
| 8 | Lift up your arms and don't let me pull them away from you | 27 | Give a thumbs-up with both hands |
| 9 | Push me away | 28 | Touch each finger to your thumb as fast as you can |
| 10 | Lift your arms out to your sides and don't let me push them down | 29 | Trace a circle in the air with both hands |
| 11 | NIHSS LOC questions | 30 | Hold your arms out straight; palms down |
| 12 | Make a fist | 31 | Flip your palms up and down five times |
| 13 | Release your fist | 32 | Cross your arms over each other five times |
| 14 | Best gaze; visual loss and smile | 33 | Lift both hands above your head and bring them back down |
| 15 | Lift your arms up and hold them for 10 seconds | 34 | Lift your arms out to your sides and do five small arm circles |
| 16 | Lift your legs up and hold them for 5 seconds | 35 | Cycle your fists in front of you |
| 17 | Touch your finger to your nose, then touch my finger | 36 | Stand up from the table and push the chair in |
| 18 | Sensory; language; dysarthria; extinction | 37 | Put on your jacket |
| 19 | Walk to the chair and sit down | 38 | Untie your shoes and tie them up again |

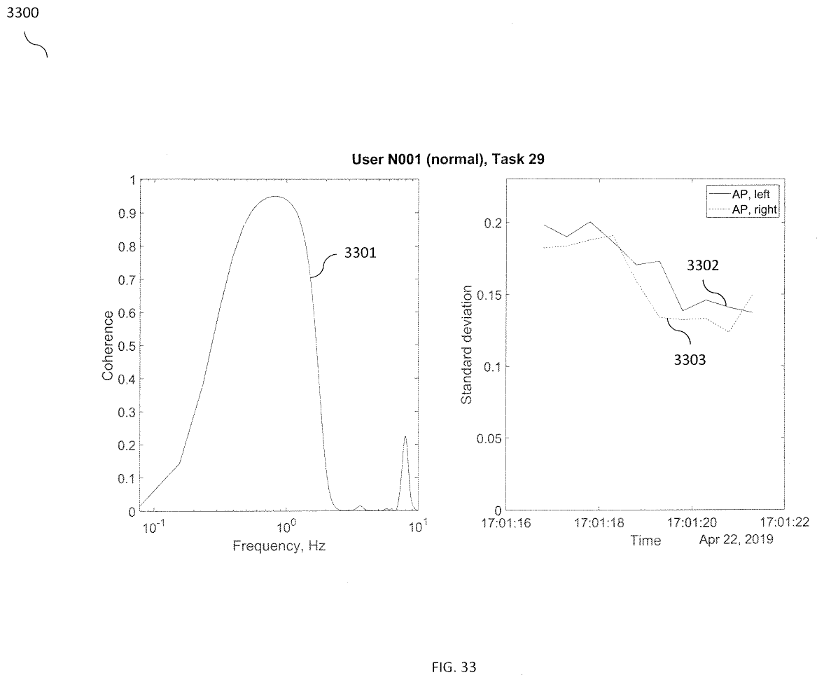

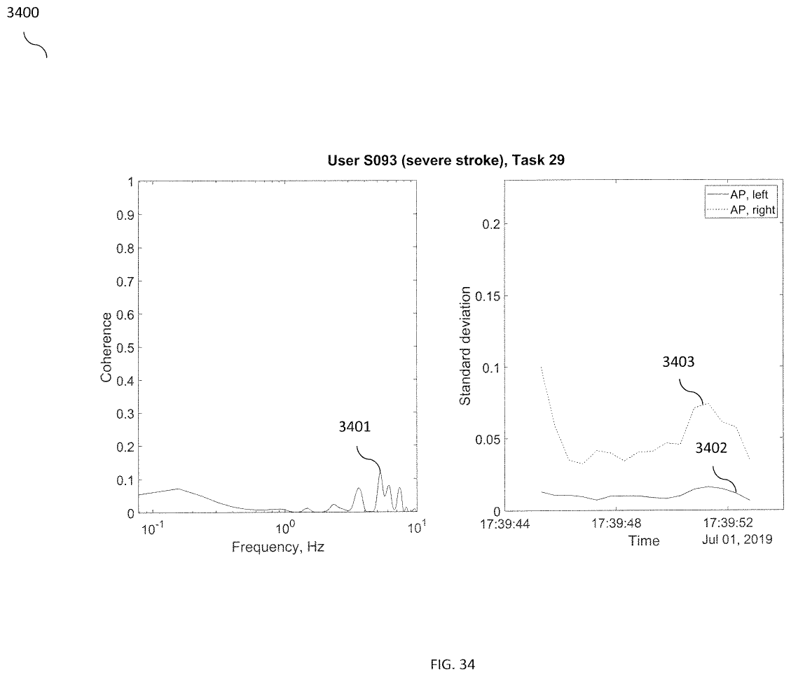

FIG. 33, a set of 2D planes 3300, shows signal features for data from a normal subject. In this example, the features are the cross-limb coherence 3301 and the standard deviations 3302, 3303 of the two signals shown in FIG. 31. As seen in FIG. 33, the maximum coherence over all frequencies is close to its theoretical maximum 1.0, while the frequency of the maximum coherence is easily identifiable. The standard deviations 3302, 3303 of both signals are considerable. As another example, FIG. 34, a set of 2D planes 3400, shows the same features but now in the case of data from a subject with stroke. In this example, the cross-limb coherence 3401 takes on very small values, with a maximum value of 0.2. The standard deviation of the signal from the stroke-affected side 3402 is less than that from the healthy limb 3403.

Once the observation table is formed, the machine learning model 1211 is trained 1212 and validated for classification. Two different classification problems are considered. First, classifying subjects into two classes (normal or stroke). Second, classifying subjects into multiple classes (a multinomial output, with the class labels such as normal and mild, moderate, and severe stroke). In some implementations the statistical learning method used for the training 1212 of the machine learning models 1211 can be a support vector machine method. While training, for every user from the observation table the machine learning model provides a prediction and compares the output with the true classification label, so that the classification accuracy of the machine learning model can be determined from the resulting confusion matrix. For every new observation representing the ability of a new user to perform the prescribed battery of tasks, the machine learning model provides a classification of the user on the scale from normal to stroke.

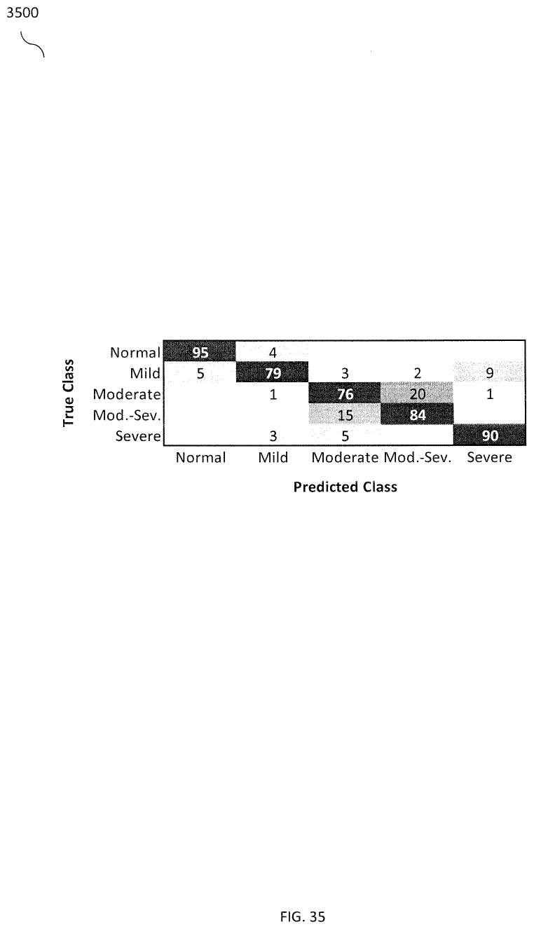

FIG. 35, confusion matrix 3500, shows an example of multinomial task-driven classification into five classes: normal, mild, moderate, moderate-to-severe, and severe, corresponding to motor deficit levels none, 1, 2, 3 and 4, respectively. The dataset was acquired in three locations at two institutions, the Maplewood Senior Living Center (MSL) and the Yale New Haven Hospital Stroke Center (YNHH). The normal subjects (control cohort, 22 subjects) were recruited from the MSL. The stroke subjects (test cohort, 61 subjects) were recruited in the stroke ward at YNHH immediately after admission for stroke (52 subjects) and in the YNHH Emergency Department (9 additional subjects) being suspected of having a stroke but before a clinical diagnosis was made; all these individuals were subsequently diagnosed with stroke. In this example, a combination of three tasks out of the total battery of 38 tasks was used to form the observation table. The numbers on the main diagonal of the confusion matrix are the percentage of true classifications (e.g., subjects with severe stroke were correctly classified as having a severe stroke in 90% of the observations), while the numbers in the other cells show percentages of misclassifications (e.g. moderate-to-severe stroke subjects were classified as moderate strokes in 15% of observations). For determining an optimal number of tasks to be used, the machine learning model 1211 was trained and validated on all possible 1- to 5-task combinations 1403 as illustrated in FIG. 14, and eventually the number of tasks producing the most accurate classification was used to classify new subjects (unseen data).

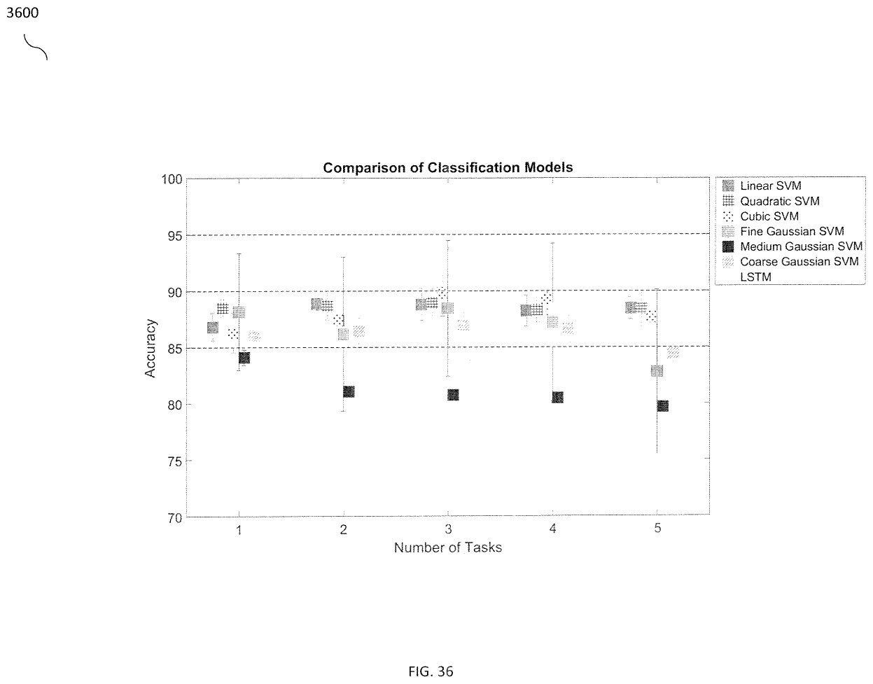

As another example from the same data as above, FIG. 36, 2D plot 3600, illustrates a comparison of the performance of 1- to 5-task machine learning models with different kernels in case of binary classification of subjects as normal or stroke. While the instances of 1 task or 2 tasks leave room for improvement, an increase in the number of tasks beyond 3 tasks leads to a slight loss of accuracy. This suggests that 3-task observation tables are optimal for building machine learning models for the task-based approach to classify subjects as normal or stroke.

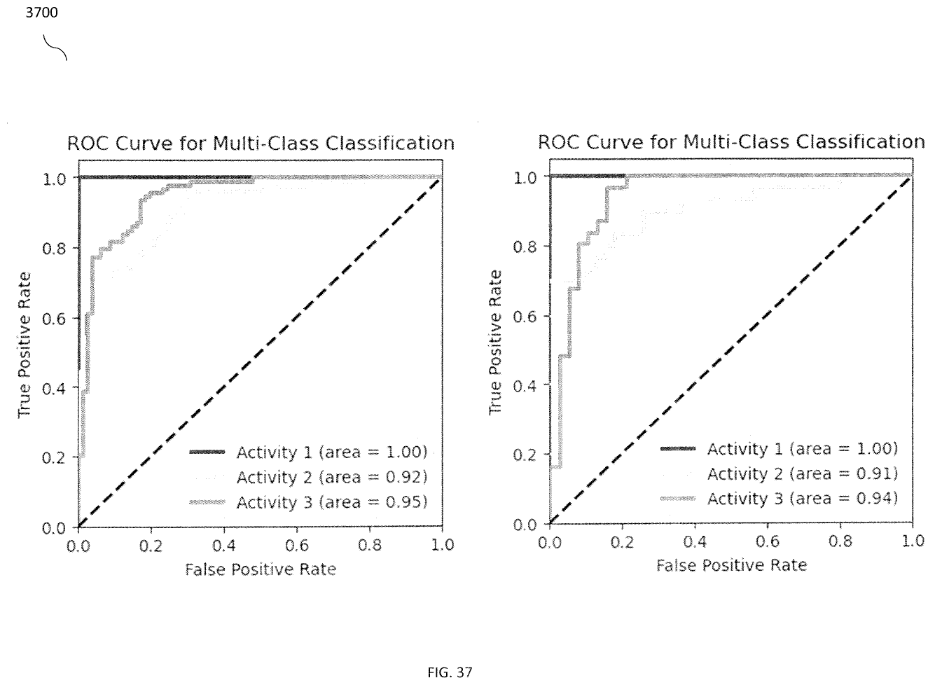

FIG. 37, a set of planes 3700, shows the detection of activities of daily living (ADLs). As seen in FIG. 37, left, when both healthy controls and stroke subjects are included, the accuracy of detection of ADLs is 87.4%, with average precision of 88%, average recall of 91% and average F1 score of 89%. When only the healthy control group is considered as shown in FIG. 37, right, the accuracy is 86.9%, with average precision of 88%, average recall of 90% and average F1 score of 89%.

### V. PATIENT MANAGEMENT SYSTEM

The patient management system 46 manages the interaction with the patient, caretaker and the emergency response system based on the data developed by the acquisition system 40 and the information developed by the analysis system 44. In normal function the patient management system has been designed to transmit information on data acquisition status to the patient. Along with this, the patient management system may send general information on stroke, cardiovascular health, and wellbeing to the patient through the smartphone.

If a change indicative of patient specific alert condition is identified the patient management system 46 performs various functions. In particular, and most importantly, the patient management system 46 directly interacts with the patient 100 to notify the patient 100 that a patient-specific alert condition has been detected. In accordance with a preferred embodiment, and as mentioned above, the patient is contacted via the communication device 10d, which in accordance with a preferred embodiment may be the smartphone 35 that is also functioning as the data transmission device 16.

The first contact with the patient 100 is used to determine if there are any untoward situations which have arisen, which resulted in the alert condition. This first contact is used to rule out conditions such as non-working body worn sensors 12. The patient 100 may be asked to perform a standard movement task. This standard movement task could be as simple as standing up and raising each arm. This interaction allows the operator of the system 10 to determine the responsiveness of the patient 100, and score their response to a standard test, akin to a neurological exam. Additional evaluations may include analysis of audio and of facial muscles to detect changes reflective of stroke.

Once it is determined that further action is necessary or if the system 10 fails to achieve interaction with the patient 100, the patient's caretaker(s) 102 is notified. The caretaker(s) is provided with detailed information on patient location, alert condition, the time of the attempt to interact with the patient, and the result of the attempt to interact with the patient. If the patient is outside a facility, then the patient management system 46 also, optionally, provides the caretaker(s) 102 with information on the nearest facility where the patient can be taken, and alert the staff at the facility as to the condition of the patient, and the alert condition which was generated. Optionally, patient management system may raise an alarm and the patient is automatically connected to the emergency system for transport to the nearest Neurocritical Care Unit or emergency room.

### VI. SUMMARY

The system 10 brings together expertise in biomaterials, sensors, electronics, real-time monitoring, time-series analysis, cloud-based analytics, stroke, and emergency neurology, to provide a real-time monitoring system for rapid transmission of acute stroke systems. These efforts rapidly set the stage for use in patients, wireless communication of potential stroke syndromes in real-time, and activation of acute stroke protocols. The system has been described, in part for application for ischemic stroke, but it can be appreciated that it is equally applicable for both ischemic and hemorrhagic stroke. The result is a product ready for immediate use where there is a clear and urgent need to improve the delivery of care for patients with stroke. The analysis system uses a number of measures to quantify signal magnitude, variability, complexity, and interrelation of the multivariate signals. These are estimated as time-series measures of NLE, ApEn, DFA, spectral entropy and coherence of multi-sensor data. These features are submitted to machine learning models to detect activities of daily living, strokes and determine the severity of a detected stroke. It is appreciated that the time-series analysis measures employed in accordance with the present invention could be supplemented by adding any number of other direct or indirect measures to improve the performance of the analysis system.

While limb activity data is the primary measure employed in the analysis method, other sensor modalities, for example, audio, video, heart rate, temperature, etc., may be employed in identifying the onset of a stroke. The system has been described, in part for application for ischemic stroke, but it can be appreciated that it is equally applicable for both ischemic and hemorrhagic stroke. The result is a product ready for immediate use where there is a clear and urgent need to improve the delivery of care for patients with stroke.

The system is also applicable for the detection and quantification of focal deficits which result from acute central nervous system injury or injury in general. It is appreciated that the measurements and analysis described allow quantification of focal deficits due to injury and provide a quantitative marker for both normal function and the loss of function due to injury. Such quantification is invaluable for rehabilitation. That is, in the event that a patient 100 has a change in limb activity introduced by a stroke, the previously established patient specific limb activity signatures can also be used to quantify the degree of success in restoring function during rehabilitation. This quantification may be more broadly applied as well. It is also contemplated that the present system 10 may be applied as a surrogate measure for overall well-being by quantifying normal levels of activity for an individual and detecting changes from these levels. For example, it is contemplated the present system 10 may be applied in conjunction with the treatment and detection of neurological and psychiatric illnesses such as psychoses, depression, post-traumatic stress-disorder, muscular sclerosis, and Parkinsonism.

The system 10 may also be used to quantify the neurological exam. Here, an individual is asked to sit, stand and perform stereotyped movements, and the system 10 documents and quantifies the limb activity, and compares them against a normative database to allow a quantitative comparison to a normal population. This examination can be performed at a remote location, facilitating telemedicine. In a second extension, the system 10 is embedded within a game which can be played on smartphones and on gaming devices. This allows the quantitative determination of limb activity while an individual is playing a game, and the detection of subtle movement disorder through game playing.

While the preferred embodiments have been shown and described, it will be understood that there is no intent to limit the invention by such disclosure, but rather, is intended to cover all modifications and alternate constructions falling within the spirit and scope of the invention.

1. A real-time automated system to diagnose or detect stroke and engage the patient, care-takers, emergency medical system and stroke neurologists in the management of this condition, comprising:
at least one body worn sensor continuously measuring natural limb activity;
a patient worn data transmission device conveying the measurements to a real-time data processing system that identifies patient specific alert conditions and determines solutions for acting upon needs of the patient.

2. The system according to claim 1, wherein the data processing system establishes patient specific limb activity signature through the aggregation of continuously sampled data acquired over minutes, hours, days, weeks, and months.

3. The system according to claim 1, further including a plurality of body worn sensors shaped and dimensioned to be worn on limbs of the patient.

4. The system according to claim 1, wherein the at least one body worn sensor includes a motion tracking device.

5. The system according to claim 1, wherein the data processing system identifies treatment protocols.

6. The system according to claim 5, wherein the treatment protocols include activation of the emergency medical response system, transport of the patient to the nearest Neurocritical Care Unit or emergency room for rapid evaluation and treatment.

7. The system according to claim 1, wherein the data processing system includes an acquisition system, an analysis system, and a patient management system.

8. The system according to claim 1, wherein the analysis system continuously processes limb activity and sensor data, and determines, in comparison to a previously determined patient specific limb activity signature if the current limb movements of the patient are within expected parameters.

9. The system according to claim 1, wherein the data processing system operates with an understanding that greater severity of stroke is associated with greater resultant loss in richness of movement signals.

10. The system according to claim 1, wherein the data processing system determines a degree of deficit resulting from a stroke.

11. The system according to claim 1, further including a machine learning model determining a degree of motor deficit induced by a stroke as reflected by changes in time-series measures of signal magnitude, variability, complexity, and interrelation.

12. The system according to claim 1, wherein the system operates in two distinct modes, a first mode continuously monitoring subject activity and a second mode evaluating short duration data segments when a subject is performing prescribed movement tasks.

13. The system according to claim 1, further including a computational method and machine learning models.

14. The system according to claim 13, wherein the computational method and the machine learning models operate in two different modes, analyzing continuous, long-term monitoring sensor data or analyzing finite, task-specific sensor data.

15. The system according to claim 13, wherein the computational method estimates features by processing data, and raw data and the features are input into the machine learning models for training to produce optimized machine learning models and into the optimized machine learning models to classify a subject state.

16. The system according to claim 15, wherein the machine leaning models identify activities of daily living, distinguish between normal and stroke states, and determine severity of a stroke.

17. The system according to claim 15, wherein the system tracks an ensemble of diffusion geometry measurements and other time-series measures of signal magnitude, variability, complexity, and interrelation determined from measurements of the body-worn sensor.

18. The system according to claim 15, wherein the data are analyzed using diffusion maps, a manifold-learning machine learning (ML) method, and time-series analysis measures.

19. The system according to claim 18, wherein the time-series analysis measures comprise non-linear energy (NLE) as a measure of signal magnitude, approximate entropy (ApEn), standard deviation, detrended fluctuation analysis (DFA) and spectral entropy as measures of signal variability and complexity, and coherence and cross-ApEn as measures of interrelationship.

20. The system according to claim 13, wherein multiple machine learning models are created for detecting activities of daily living, detecting stroke, determining severity of stroke from continuous data, and determining severity of stroke from task-based data.

21. The system according to claim 1, wherein motor deficit assessment is achieved using a deep neural networks to process sequential data to detect motor asymmetry or motor deficit difference between affected and unaffected arms.

22. The system according to claim 1, wherein the system detects stroke and activities of daily living, and quantifies severity of motor deficit from tasks.

23. The system according to claim 1, wherein synchronization is achieved by considering a smartphone clock to be a master clock and constantly measure and correct wearable device clock drift with respect to the smartphone clock.
